(19) United States
(12) Patent Application Publication
(10) Pub. No.: US 2019/0300885 A1
(43) Pub. Date: Oct. 3, 2019

(54) A NOVEL ROLE FOR TERMINAL RNA URIDYLATION AND RNA TURNOVER IN ONCOGENESIS

(71) Applicant: Children's Medical Center Corporation, Boston, MA (US)

(72) Inventors: George Q. Daley, Cambridge, MA (US); Kaloyan Tsanov, Cambridge, MA (US); Daniel S. Pearson, Boston, MA (US)

(73) Assignee: Children's Medical Center Corporation, Boston, MA (US)

(21) Appl. No.: 16/308,196

(22) PCT Filed: Jun. 7, 2017
(86) PCT No.: PCT/US17/36436

§ 371 Date: Dec. 7, 2018

Related U.S. Application Data
(60) Provisional application No. 62/347,048, filed on Jun. 7, 2016.

(51) Int. Cl. C12N 15/113; A61K 31/433; A61K 31/275; A61K 31/555; A61K 31/41; A61K 31/7135; A61K 31/10; A61P 35/00

(52) U.S. Cl. CPC C12N 15/1137; A61K 31/433; A61K 31/275; A61K 31/555; A61K 31/41; C12N 2310/20; A61K 31/10; C12Y 207/07052; A61P 35/00; C12N 2310/14; A61K 31/7135

(57) ABSTRACT

Described herein is a LIN28-independent role of TUTases in oncogenesis. Provided herein are compositions and methods for treating cancer via inhibition of TUTases. TUTase depletion also sensitizes the cells to disruptions in RNA metabolism and/or protein metabolism. Thus, further provided herein are strategies of combination therapy, combining TUTase inhibitors, agents that disrupt RNA metabolism, and agents that disrupt protein metabolism, to treat cancer.

### RELATED APPLICATIONS

This application claims the benefit under 35 U.S.C. § 119(e) of U.S. provisional application No. 62/347,048, filed Jun. 7, 2016, which is incorporated by reference herein in its entirety.

### GOVERNMENT SUPPORT

This disclosure was made with government support under 5R01GM107536 and T32GM007753 awarded by the National Institutes of Health. The government has certain rights in the disclosure.

### BACKGROUND

3′-terminal uridylyl transferases (TUTases) have been indicated to play a role in microRNA (e.g., let-7 miRNA) biogenesis in a LIN-28/AB-dependent manner. The uridylation of pre-let-7 by TUTases (e.g., ZCCHC6 or ZCCHC11 in mammalian cells) leads to its degradation and in turn the repression of mature let-7 miRNA. A number of cancers have been linked with LIN28A/B mediated repression of let-7 miRNA. However, a LIN28A/B-independent role of TUTase in oncogenesis has not been described.

### SUMMARY

The present disclosure provides compositions and methods to treat cancer (e.g., cancer that does not express LIN28A/B) via inhibition of 3′ terminal uridylyl transferases (TUTases). As shown herein, mammalian TUTases (e.g., ZCCHC6 and ZCCHC11) regulate mRNA uridylation and turnover in a LIN28-independent and microRNA-independent manner. Further, TUTases are overexpressed in diverse types of cancers and depletion of TUTases inhibits cancer growth. Accordingly, described herein are methods of treating cancer using TUTase inhibitors, and combination therapy based on the finding that TUTases-depleted cells are sensitized to disruptions in RNA and/or protein metabolism.

Some aspects of the present disclosure provide methods of treating cancer, comprising administering to a subject in need thereof a composition comprising a therapeutically effective amount of a terminal uridylyl transferase (TUTase) inhibitor to treat the cancer, wherein the cancer does not express LIN28A/B.

In some embodiments, the TUTase is ZCCHC11 or ZCCHC6.

In some embodiments, the TUTase inhibitor is a small molecule, an oligonucleotide, an antibody, or an antibody fragment. In some embodiments, the TUTase inhibitor inhibits the TUTase's enzymatic activity. In some embodiments, the TUTase inhibitor is selected from the group consisting of: SCH 202676 hydrobromide, Tryphostin 47, FPA 124, Ebselen, Aurothioglucose hydrate, and IPA-3.

In some embodiments, the TUTase inhibitor reduces TUTase expression. In some embodiments, the TUTase inhibitor is RNAi targeting ZCCHC11 or ZCCHC6. In some embodiments, the TUTase inhibitor is a vector that co-expresses a Cas9 nuclease, and a guide RNA that targets the Cas9 to the ZCCHC11 or ZCCHC6 gene, whereby the ZCCHC6 or ZCCHC11 gene is cleaved by the Cas9.

In some embodiments, the antibody or the antibody fragment is specific to ZCCHC11 or ZCCHC6.

In some embodiments, the composition further comprises a therapeutically effective amount of an agent that disrupts RNA metabolism. In some embodiments, the agent that disrupts RNA metabolism inhibits nucleotide synthesis or metabolism. In some embodiments, the agent that disrupts RNA metabolism is a purine and pyrimidine antimetabolite. In some embodiments, the purine and pyrimidine antimetabolite is a 5′ fluoropyrimidine. In some embodiments, the 5′ fluoropyrimidine is 5-fluorouracil (5-FU), Ftorafur, or uracil tegafur (UFT). In some embodiments, the 5′ fluoropyrimidine is 5-FU. In some embodiments, the purine and pyrimidine antimetabolite is selected from the group consisting of: 6-Mercaptopurine, Azathioprine, Fludarabine, Decitabine, Nelarabine, Clofarabine, Vidaza, Capecitabine, Gemcitabine, Pentostatin, Floxuridine, Cytarabine, and 6-thioguanine. In some embodiments, the agent that disrupts RNA metabolism is an antifolate. In some embodiments, the antifolate is selected from the group consisting of Methotrexate, Pemetrexed, Nolatrexed, Raltitrexed, and ZD9331.

In some embodiments, the composition further comprises an agent that disrupts protein metabolism. In some embodiments, the agent that disrupts protein metabolism inhibits protein turnover. In some embodiments, the agent that disrupts protein metabolism is a proteasome inhibitor. In some embodiments, the proteasome inhibitor is selected from the group consisting of: bortezomib, Lxazomib, Carfilzomib, Oprozomib (ONX-0912), Delanzomib (CEP-18770), Marizomib (salinosporamide A), Lactacystin, Disulfiram Epigallocatechin-3-gallate, Epoxomicin, and MG132Beta-hydroxy beta-methylbutyrate. In some embodiments, the proteasome inhibitor is bortezomib.

In some embodiments, the agent that disrupts protein metabolism is a PI3K/mTOR inhibitor. In some embodiments, the PIK3/mTOR inhibitor is rapamycin or a rapalog. In some embodiments, the rapalog is selected from the group consisting of: Sirolimus, Temsirolimus, Everolimus, and Deforolimus. In some embodiments, the PIK3/mTOR inhibitor is an ATP-competitive mTOR kinase inhibitor. In some embodiments, the ATP-competitive mTOR kinase inhibitor is Torin1 or Torin2.

In some embodiments, the composition inhibits cancer cell growth and reduces tumor size. In some embodiments, the composition prevents metastasis.

In some embodiments, the composition is administered via injection to the cancer. In some embodiments, the composition is administered systemically.

In some embodiments, the subject is a mammal. In some embodiments, the mammal is a human. In some embodiments, the mammal is a rodent. In some embodiments, the rodent is a rat. In some embodiments, the rodent is a mouse.

In some embodiments, the TUTase inhibitor increases mRNA half-life in cancer cells, compared to without the TUTase inhibitor.

Other aspects of the present disclosure provide methods of treating cancer, comprising administering to a subject in need thereof a composition comprising a therapeutically effective amount of a terminal uridylyl transferase (TUTase) inhibitor and an agent that disrupts RNA metabolism, to treat the cancer.

In some embodiments, the TUTase is ZCCHC11 or ZCCHC6.

In some embodiments, the TUTase inhibitor is a small molecule, an oligonucleotide, an antibody, or an antibody fragment. In some embodiments, the TUTase inhibitor inhibits the TUTase's enzymatic activity. In some embodiments, the TUTase inhibitor is selected from the group consisting of: SCH 202676 hydrobromide, Tryphostin 47, FPA 124, Ebselen, Aurothioglucose hydrate, and IPA-3. In some embodiments, the TUTase inhibitor reduces TUTase expression. In some embodiments, the TUTase inhibitor is RNAi targeting ZCCHC11 or ZCCHC6. In some embodiments, the TUTase inhibitor is a vector that co-expresses a Cas9 nuclease, and a guide RNA that targets the Cas9 to the ZCCHC11 or ZCCHC6 gene, whereby the ZCCHC6 or ZCCHC11 gene is cleaved by the Cas9. In some embodiments, the antibody or the antibody fragment is specific to ZCCHC11 or ZCCHC6.

In some embodiments, the agent that disrupts RNA metabolism inhibits nucleotide synthesis or metabolism. In some embodiments, the agent that disrupts RNA metabolism is a purine and pyrimidine antimetabolite. In some embodiments, the purine and pyrimidine antimetabolite is a 5′ fluoropyrimidine. In some embodiments, the 5′ fluoropyrimidine is 5-fluorouracil (5-FU), Ftorafur, or uracil tegafur (UFT). In some embodiments, the 5′ fluoropyrimidine is 5-FU. In some embodiments, the purine and pyrimidine antimetabolite is selected from the group consisting of: 6-Mercaptopurine, Azathioprine, Fludarabine, Decitabine, Nelarabine, Clofarabine, Vidaza, Capecitabine, Gemcitabine, Pentostatin, Floxuridine, Cytarabine, and 6-thioguanine. In some embodiments, the agent that disrupts RNA metabolism is an antifolate. In some embodiments, the antifolate is selected from the group consisting of Methotrexate, Pemetrexed, Nolatrexed, Raltitrexed, and ZD9331.

In some embodiments, the composition further comprises an agent that disrupts protein metabolism. In some embodiments, the agent that disrupts protein metabolism inhibits protein turnover. In some embodiments, the agent that disrupts protein metabolism is a proteasome inhibitor. In some embodiments, the proteasome inhibitor is selected from the group consisting of: bortezomib, Lxazomib, Carfilzomib, Oprozomib (ONX-0912), Delanzomib (CEP-18770), Marizomib (salinosporamide A), Lactacystin, Disulfiram Epigallocatechin-3-gallate, Epoxomicin, and MG132Beta-hydroxy beta-methylbutyrate. In some embodiments, the proteasome inhibitor is bortezomib.

In some embodiments, the agent that disrupts protein metabolism is a PI3K/mTOR inhibitor. In some embodiments, the PIK3/mTOR inhibitor is rapamycin or a rapalog. In some embodiments, wherein the rapalog is selected from the group consisting of: Sirolimus, Temsirolimus, Everolimus, and Deforolimus. In some embodiments, the PIK3/mTOR inhibitor is an ATP-competitive mTOR kinase inhibitor. In some embodiments, the ATP-competitive mTOR kinase inhibitor is Torin1 or Torin2.

In some embodiments, the cancer does not express LIN28A/B.

In some embodiments, the composition inhibits cancer cell growth and reduces tumor size. In some embodiments, the composition prevents metastasis.

In some embodiments, the composition is administered via injection to the cancer. In some embodiments, the composition is administered systemically.

In some embodiments, the subject is a mammal. In some embodiments, the mammal is a human. In some embodiments, the mammal is a rodent. In some embodiments, the rodent is a rat. In some embodiments, the rodent is a mouse.

In some embodiments, the TUTase inhibitor increases mRNA half-life in cancer cells, compared to without the TUTase inhibitor.

Further provided herein are pharmaceutical compositions comprising a terminal uridylyl transferase (TUTase) inhibitor for treating cancer, wherein the cancer does not express LIN28A/B.

Further provided herein are pharmaceutical compositions comprising a terminal uridylyl transferase (TUTase) inhibitor and an agent that disrupts RNA metabolism, for treating cancer.

In some embodiments, the agent that disrupts RNA metabolism is 5-fluorouracil. In some embodiments, the composition further comprises an agent that disrupts protein metabolism. In some embodiments, the agent that disrupts protein metabolism is a proteasome inhibitor. In some embodiments, the proteasome inhibitor is bortezomib. In some embodiments, the agent that disrupts protein metabolism is a PIK3/mTOR inhibitor. In some embodiments, the composition further comprises a pharmaceutically acceptable carrier.

Each of the limitations of the disclosure can encompass various embodiments of the disclosure. It is, therefore, anticipated that each of the limitations of the disclosure involving any one element or combinations of elements can be included in each aspect of the disclosure. This disclosure is not limited in its application to the details of construction and the arrangement of components set forth in the following description or illustrated in the drawings. The disclosure is capable of other embodiments and of being practiced or of being carried out in various ways.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein.

FIGS. 1A-1B. ZCCHC6/11 are expressed in select normal adult human tissues. FIG. 1A shows a Western blot analysis of lysates from 14 healthy human tissues. Expected sizes of major ZCCHC6 and ZCCHC11 isoforms are indicated on the right. Asterisks denote LIN28A/B-expressing tissues. FIG. 1B shows RNA-seq analysis of samples from 30 healthy human tissues. The different lines represent distinct predicted splice isoforms of ZCCHC6 and ZCCHC11, as indicated on the right. Data were obtained from the GTEx Portal (gtexportal.org).

FIGS. 2A-2B. ZCCHC6/11 are highly expressed in diverse types of cancer. FIG. 2A shows ZCCHC6/11 mRNA levels (Z-score) in human patient samples from different types of tumors. Heat map depicts aggregated expression data while histograms show data grouped by tumor type. Each bar represents an individual patient. Data were obtained from the cBio Portal (cbioportal.org). FIG. 2B shows a Western blot analysis of 30 human cancer cell lines representing different types of cancer. BJ1 fibroblasts are included as a normal tissue reference.

FIGS. 3A-3E. TUTase overexpression promotes oncogenic transformation. FIG. 3A shows colony formation in soft agar of NIH3T3 cells harboring lentiviral overexpression of GFP, wild-type ZCCHC6 (Z6(WT)), catalytic-null ZCCHC6 (Z6(DADA)), or mutant KRAS (RAS(G12V)). Representative images of three independent experiments are shown. Magnification 4×. FIG. 3B presents a quantification of colony formation assay from FIG. 3A. Values represent the sum of counts from four independent fields of view. Error bars indicate SEM (n=3); *P<0.05, **P<0.01. FIG. 3C shows a Western blot analysis of cells analyzed in FIGS. 3A-3B. FIG. 3D shows colony formation in soft agar of NIH3T3 cells harboring lentiviral overexpression of the indicated constructs. RAS=RAS(G12V), MYC=C-MYC. Representative images of three independent experiments are shown. Magnification 4×. FIG. 3E shows a quantification of colony formation assay from FIG. 3D. Values represent the sum of counts from four independent fields of view. Error bars indicate SEM (n=3); n.s.=non-significant (P>0.05), *P<0.05, **P<0.01.

FIGS. 4A-4G. TUTase depletion impairs growth in diverse cancer cell types. FIG. 4A presents a summary of results from a ZCCHC6/11 siRNA screen of a panel of 19 cell lines representing six different types of cancer. Percentage of cell lines with (dark grey) or without (light grey) a growth impairment phenotype upon dual TUTase knockdown is displayed. FIG. 4B shows a cell proliferation analysis of representative TUTase-dependent cell lines. Cells were reverse transfected with siRNAs against ZCCHC6 and ZCCHC11 (siZ6+Z11) or a negative control (siNC), counted 24 h later (d0), and re-counted another 96 h later (d4). Error bars indicate SEM (n=3); *P<0.05, **P<0.01. FIG. 4C shows focus formation of HCT116 cells after CRISPR/Cas9-mediated knockout using a ZCCHC6-targeting sgRNA (KO) or a GFP-targeting control sgRNA (WT). Error bars indicate SEM (n=3); **P<0.01. Representative images (crystal violet stain) are shown on the right. Magnification 4×. FIG. 4D shows colony formation in soft agar of HCT116 cells after CRISPR/Cas9-mediated knockout using a ZCCHC6-targeting sgRNA (KO) or a GFP-targeting control sgRNA (WT). Error bars indicate SEM (n=3); *P<0.05. Representative images (crystal violet stain) are shown on the right. Magnification 4×. FIG. 4E shows growth curves of subcutaneous xenografts generated from HCT116 cells after CRISPR/Cas9-mediated knockout using a ZCCHC6-targeting sgRNA (gZ6; bottom line) or a GFP-targeting control sgRNA (gGFP; top line). Days after injection are indicated on the x-axis. Error bars indicate SEM (n=7); ***P<0.001 (linear regression test). FIG. 4F shows tumor weight measurements of xenografts from FIG. 4E at the time of harvest (day 43). Error bars indicate SEM (n=7); **P<0.01. Representative images of the tumors are shown on the right. FIG. 4G shows a Western blot analysis of xenograft tumors from FIG. 4E at the time of harvest (day 43).

FIGS. 5A-5B. TUTase loss sensitizes cancer cells to disruption of RNA metabolism. FIG. 5A shows a summary of utilized drugs and their mechanism of action (table on top), and schematic of the experimental workflow (bottom). FIG. 5B shows growth curves of HCT116 cells after CRISPR/Cas9-mediated knockout using a ZCCHC6-targeting sgRNA (KO) or a GFP-targeting control sgRNA (WT) under treatment with different concentrations of the indicated drugs, as indicated in FIG. 5A. Error bars indicate SEM (n=3); *P<0.05, **P<0.01.

FIGS. 6A-6F. TUTases uridylate mRNAs and enhance their turnover in cancer cells. FIG. 6A provides TAIL-seq data (n=3) showing mRNA uridylation frequency in HCT116 cells after ZCCHC6 knockdown (siZ6). A scrambled siRNA served as a negative control (siNC). Fraction of mRNA reads among the total poly(A)+ reads is shown in each poly(A) tail size range. Light grey refers to non-U reads, while darker shades represent uridylated reads, as indicated. Schematic of the experimental workflow is shown on top. FIG. 6B provides RNA-seq data (n=3) showing distribution of mRNA expression levels after ZCCHC6 knockdown. The x-axis depicts individual genes and the y-axis indicates corresponding Log 2 fold-change in their mRNA levels. Libraries were prepared using two alternative protocols, as indicated. Correlation between data obtained from the two protocols is shown on the bottom. FIG. 6C shows the correlation between uridylation frequency (using HeLa data shown in FIGS. 12A-12D) and mRNA half-life (using HeLa data from Tani et al., 2012) on a per gene basis. FIG. 6D shows genome-wide mRNA half-life measurements using actinomycin D chase coupled with RiboZero RNA-seq (n=3). The schematic on top indicates experimental workflow, and the graphs on bottom depict average mRNA half-life after ZCCHC6 knockdown (siZ6) vs. control (siNC), on a per gene basis (left) or aggregated (right). FIG. 6E shows qRT-PCR validation (n=3) of half-life measurements from FIG. 6D. Data on four representative genes are shown. FIG. 6F shows qRT-PCR analysis (n=3) of the half-lives of short non-coding RNAs after ZCCHC6 knockdown. The experiment was performed essentially as shown in FIG. 6D, with the addition of a 12 h time point.

FIGS. 7A-7C. In vivo assessment of the requirement for TUTases in colorectal tumorigenesis. FIG. 7A shows a Western blot analysis of tissue lysates from mouse models of colorectal cancer. Both intestinal (top) and colonic (bottom) tumors were analyzed. Samples were obtained from the standard ApcMin model and the more aggressive ApcMin+Lin28a and ApcMin+LIN28B models. Normal mucosa from wild-type animals served as a control. Each lane represents an independent tumor. FIG. 7B shows a Western blot analysis of tissue lysates from human colorectal cancer patients. Normal mucosa from matched (right) and unmatched (left) patients served as a control. Each lane represents an independent tumor, with matched samples being grouped with a bracket. FIG. 7C shows the breeding strategy for assessment of the TUTase requirement for colorectal tumorigenesis in the ApcMin mouse model.

FIGS. 8A-8B. Generation of doxycycline-inducible TUTase overexpressing ESCs. FIG. 8A shows a schematic of doxycycline-inducible ZCCHC6/11 (iZCCHC6/11) alleles. FIG. 8B shows a qRT-PCR analysis of iZCCHC6 (left) and iZCCHC11 (right) ESC clones.

FIGS. 9A-9D. LIN28B and ZCCHC6/TUT7 interact in the cytoplasm in an RNA-dependent manner. FIG. 9A is a Venn diagram depicting the intersection between Lin28a and LIN28B protein partners. FIG. 9B shows mass spectrometry analysis of LIN28B purified from BE2C cells. FIG. 9C shows a Western blot validation of the mass spectrometry data. FIG. 9D shows a Western blot analysis of subcellular fractions from BE2C cells. Tubulin and fibrillarin serve as cytoplasmic and nuclear markers, respectively. WCE=whole cell extract; Cyt=cytoplasm; Nuc=nucleus.

FIGS. 10A-10C. ZCCHC6/11 depletion does not universally alter mature let-7 levels. Mature let-7 levels after knockdown of LIN28B with two independent siRNAs (siB-2 and siB-3), ZCCHC6 (siZ6), ZCCHC11 (siZ11), or both ZCCHC6 and ZCCHC11 (siZ6+11) in HepG2 (FIG. 10A), BE2C (FIG. 10B), and 293T (FIG. 10C) cells are shown. Western blot validation of knockdowns is shown on the bottom of each figure, respectively. Analysis was performed 96 h after siRNA transfection. Error bars indicate SEM (n=3); *P<0.05, **P<0.01.

FIGS. 11A-11B. ZCCHC6/11 are rarely amplified, deleted, or mutated in human tumors. FIG. 11A shows the frequency of mutations, deletions, and amplifications of ZCCHC6/11 in human patient samples from different types of tumors. Each bar represents a distinct type of cancer. Data were obtained from the cBio Portal (cbioportal.org). FIG. 11B shows the frequency of specific mutations in ZCCHC6/11 in human patient samples from different types of tumors. Data were obtained from the cBio Portal (cbioportal.org).

FIGS. 12A-12B. CRISPR/Cas9-based genetic disruption confirms RNAi-based phenotypes. FIG. 12A shows a cell proliferation analysis of HCT116 cells after CRISPR/Cas9-mediated knockout of ZCCHC6 with two independent sgRNAs (KO#1 and KO#2). Two GFP-targeting sgRNAs (WT#1 and WT#2) were used as negative controls. Cells were transduced with Cas9/sgRNA lentiviral particles, selected with puromycin for five days, counted to establish starting cell numbers (d0), and re-counted after 96 h (d4). Western blot assessment of population-level knockout efficiency is shown on the bottom. Error bars indicate SEM (n=3); *P<0.05. FIG. 12B shows a cell proliferation analysis of H1299 cells after CRISPR/Cas9-mediated knockout of ZCCHC6 and ZCCHC11 (DKO). Dual GFP-targeting sgRNAs were used as a negative control (WT). The assay was performed analogously to the experiment in FIG. 12A. Western blot assessment of population-level knockout efficiency is shown on the bottom. Error bars indicate SEM (n=3); **P<0.01.

FIGS. 13A-13B. TUTase depletion impairs cancer cell growth in a LIN28 and miRNA-independent manner. Cell proliferation analysis of wild-type (FIG. 13A) and DICER^{Ex5} (FIG. 13B) HCT116 cells after knockdown of ZCCHC6 with two independent siRNAs (siZ6#1 and siZ6#2), ZCCHC11 (siZ11), or both ZCCHC6 and ZCCHC11 (siZ6#1+11 and siZ6#2+11) are shown. Cells were reverse transfected, counted 24 h later, and re-counted another 96 h later. Final counts were first normalized to starting counts for each condition and then to the control condition (siNC). Western blot assessment of knockdown efficiency is shown on the bottom of each respective figure.

FIGS. 14A-14D. TAIL-seq can quantitatively assess 3′ mRNA uridylation. FIG. 14A depicts the experimental procedure for TAIL-seq (adapted from Chang et al., 2014). FIG. 14B shows the number of mRNA reads obtained using four optimization versions (s1-s4) of the TAIL-seq protocol in HeLa cells. The bottom bar refers to reads lacking a poly(A) sequence (non-poly(A)), while the subsequent bars from bottom to top represent poly(A)+ reads that are non-uridylated (non-U), mono-uridylated (U), di-uridylated (UU), or tri- or more uridylated (U≥3), respectively. Data processing was performed as described in Chang et al. (2014), with modifications (see examples section for details). FIG. 14C shows the uridylation frequency for samples s1-s4. Fraction of mRNA reads among the total poly(A)+ reads is shown in each poly(A) tail size range. Light grey refers to non-U reads, while darker shades represent uridylated reads, as indicated. FIG. 14D presents Venn diagrams depicting the intersection of our data (s1 and s2 are used as representative samples) and public data from Chang et al. (2014).

FIG. 15. Validation of sgRNAs used in RNA decay factor screen. qRT-PCR analysis of indicated RNA decay factors in HCCT116 cells is shown. Five distinct sgRNAs (1-5) were tested. A GFP-targeted sgRNA served as a negative control. Cells were transduced with Cas9/sgRNA lentiviral particles, selected with puromycin for five days, and harvested for RNA isolation. Boxed sgRNAs were selected for use in the screen.

FIG. 16. TUTase loss has a downstream impact on protein turnover. Top, the relative rates of global mRNA translation as measured by the OP-Puromycin fluorescent assay are shown. siNC=negative control siRNA; siZ6=siZCCHC6. Error bars indicate SEM (n=3); *P<0.05. Bottom, growth curves of HCT116 cells after CRISPR/Cas9-mediated knockout using a ZCCHC6-targeting sgRNA (KO) or a GFP-targeting control sgRNA (WT) under treatment with different concentrations of bortezomib are shown.

### DETAILED DESCRIPTION OF CERTAIN EMBODIMENTS

It should be understood that this disclosure is not limited to the particular methodology, protocols, and reagents, etc., described herein and as such may vary. The terminology used herein is for the purpose of describing particular embodiments only, and is not intended to limit the scope of the present disclosure, which is defined solely by the claims.

As used herein and in the claims, the singular forms include the plural reference and vice versa unless the context clearly indicates otherwise. The term “or” is inclusive unless modified, for example, by “either.” Other than in the operating examples, or where otherwise indicated, all numbers expressing quantities of ingredients or reaction conditions used herein should be understood as modified in all instances by the term “about” “About” when used in connection with percentages means±1% unless otherwise specified.

All patents and other publications identified are expressly incorporated herein by reference for the purpose of describing and disclosing, for example, the methodologies described in such publications that might be used in connection with the present disclosure. These publications are provided solely for their disclosure prior to the filing date of the present application. Nothing in this regard should be construed as an admission that the inventors are not entitled to antedate such disclosure by virtue of prior disclosure or for any other reason. All statements as to the date or representation as to the contents of these documents is based on the information available to the applicants and does not constitute any admission as to the correctness of the dates or contents of these documents.

Unless defined otherwise, all technical and scientific terms used herein have the same meaning as those commonly understood to one of ordinary skill in the art to which this disclosure pertains. Although any known methods, devices, and materials may be used in the practice or testing of the disclosure, the methods, devices, and materials in this regard are described herein.

RNA uridylyl transferases are enzymes that catalyze the addition of a nonencoded 3′ oligoU tail to RNAs (e.g., mRNAs). In some instances, RNA uridylyl transferases are also referred to as “terminal uridylyl transferases (TUTases),” or “3′ terminal uridylyl transferases.” These terms are used interchangeably in the present disclosure.

TUTases play an important role in regulating microRNA biogenesis in the context of cancer in a LIN28-dependent manner. LIN28 is a RNA-binding protein that regulates gene expression partially via Let7 biogenesis. The Let7 family of miRNAs regulates many factors that control cell-fate decision, including oncogenes (c-myc, Ras, HMGA-2) and cell-cycle factors (CyclinD1, D2). In mammals, LIN28A and its closely related paralog LIN28B are highly expressed in pluripotent cells, where they play an important role in the maintenance of self-renewal and proliferation. For example, in US Patent Publication US20140328858, TUTases (e.g., mammalian homologous ZCCHC6 and ZCCHC11) are recruited by LIN28A to uridylate a microRNA precursor, pre-let-7. The uridylation of pre-let-7 leads to its degradation and the repression of mature let-7 microRNA, which in turn leads to the oncogenic transformation of cells and promote their growth and tumorigenicity. However, besides pre-let-7 uridylation, a direct link between TUTase-mediated RNA uridylation and oncogenesis has not previously been described or suggested.

One aspect of the present disclosure is based, at least in part, on the novel and unexpected finding that TUTase promotes oncogenesis via a pathway that is independent of LIN28A/B and microRNAs (e.g., let-7 microRNA). As described in the figures and examples of the present disclosure, knocking down TUTases (e.g., ZCCHC11 and ZCCHC6) did not have appreciable effect on cellular let-7 level, while LIN28B knockdown caused let-7 derepression, indicating that ZCCHC6 and ZCCHC11 do not universally regulate let-7 in conjunction with LIN28A/B. Instead, the TUTases appear to be playing a role in mRNA uridylation and global mRNA decay regulation, as described in Lim et al., Cell. 2014 December 4; 159(6): 1365-1376. Further, the present disclosure provides evidence that TUTases (ZCCHC6 and ZCCHC11) are overexpressed in several types of cancers (e.g., colon, lung, and liver). Induced overexpression of TUTases in normal cells can promote oncogenic transformation, while depletion of TUTases in diverse cancer types inhibited cancer cell growth. Interestingly, the correlation between TUTases and cancer are independent of LIN28A/B expression. Thus, provided herein, are data establishing TUTases as a new class of oncogenes, and strategies of exploiting the new role of TUTases in oncogenesis to treat cancer, including LIN28A/B positive or negative cancer.

Accordingly, some aspects of the present disclosure provide pharmaceutical compositions for treating cancer. Such pharmaceutical composition comprises a therapeutically effective amount of a TUTase inhibitor.

In some embodiments, the cancer is does not express LIN28A/B. A cancer that does not express LIN28A/B may also be referred to as a “LIN28-negative cancer.” To determine whether a cancer expresses LIN28A/B, one skilled in the art, e.g., a physician or a clinician, may obtain a sample of the cancer (e.g., a biopsy sample), and analyze LIN28 expression via a number of techniques that are well known in the art that may be used for protein expression analysis. Such techniques include, without limitation, western blotting, immunohistostaining assays, and qRT-PCR. See, Triboulet et al., Cell Rep. 2015 Oct. 13; 13(2): 260-266, wherein LINI28A and LIN28B expression was detected using anti-LIN28A (A177)(Cell Signaling, 3978) and anti-LIN28B (Cell Signaling, 4196). See also, Liu et al., PLoS One. 2013; 8(12): e83083, wherein LIN28 expression was analyzed in a panel of breast cancer cell lines by western blotting and by immunohistochemistry.

It is to be understood that the present disclosure does not exclude the treatment of LIN28-positive cancer, i.e., cancer associated with LIN28A/B expression. It is anticipated that the TUTase inhibitors disclosed herein will also be effective in treating LIN28A/B positive cancer.

In some embodiments, the TUTase is the mammalian homolog ZCCHC6 (also known as TUT7) or ZCCHC11 (also known as TUT4). ZCCHC6 and ZCCHC11 are both involved in microRNA (miRNA)-induced gene silencing through uridylation of deadenylated miRNA targets, and act as a suppressors of miRNA biogenesis by mediating the terminal uridylation of some miRNA precursors, including that of let-7 (pre-let-7). ZCCHC6 and ZCCHC11 are highly similar in their domain organization and activities. Their functional roles in miRNA uridylation or mRNA uridylation have been shown to be redundant.

Some aspects of the present disclosure provide TUTase inhibitors. The term “inhibition” or “inhibit” when referring to the gene expression and/or activity or protein of a TUTase, such as ZCCHC11 or ZCCHC6, or a functional domain thereof, refers to a reduction or prevention in the level of its function or a reduction of its gene expression product. The term depletion, or TUTase depletion, as used herein, refers to the depletion of TUTase expression or TUTase activity. For example, TUTase depletion may mean the TUTase is genetically knocked down or TUTase activity is inhibited, e.g., by a small molecule inhibitor.

When the TUTase enzymatic activity is inhibited, it means there is a decrease in the TUTase enzymatic activity by at least about 5%, about 10%, about 20%, about 30%, about 40%, about 50%, about 60%, about 70%, about 80%, about 90%, about 95%, about 99%, about 100% of the TUTase enzymatic activity found in the cell without the presence of the TUTase inhibitor. In one embodiment, the TUTase enzymatic activity levels are decreased by at least about 70%, about 80%, about 90%, about 95%, about 99%, about 100%.

In some embodiments, a “TUTase inhibitor” is a small molecule inhibitor that inhibits TUTase activity. For example, a recent publication by Lin et al. (RNA Biology, Volume 12, Issue 8, 2015) identified a number of small molecule inhibitors for ZCCHC11. The TUTase inhibitor that may be used in accordance with the present disclosure may be, without limitation, SCH 202676 hydrobromide, Tryphostin 47, FPA 124, Ebselen, Aurothioglucose hydrate, IPA-3, or combinations thereof. All of the small molecule TUTase inhibitors described herein are commercially available, e.g., from Sigma-Aldrich or Tocris Bioscience.

In some embodiments, a “TUTase inhibitor” is an agent that reduces the expression of TUTase. Such agent may be a nucleic acid inhibitor. Nucleic acid inhibitors of TUTase are for example, but not are limited to, RNA interference-inducing molecules (RNAi), for example but are not limited to siRNA, dsRNA, stRNA, shRNA and modified versions thereof, where the RNA interference molecule silences the gene expression of a TUTase such as ZCCHC11 or ZCCHC6. In some embodiments, the nucleic acid inhibitor is an anti-sense oligonucleic acid, or a nucleic acid analogue, for example but are not limited to DNA, RNA, peptide-nucleic acid (PNA), pseudo-complementary PNA (pc-PNA), or locked nucleic acid (LNA) and the like. In alternative embodiments, the nucleic acid is DNA or RNA, and nucleic acid analogues, for example PNA, pcPNA and LNA. A nucleic acid can be single or double stranded, and can be selected from a group comprising nucleic acid encoding a protein of interest, oligonucleotides, PNA, etc. Such nucleic acid sequences include, for example, but are not limited to, nucleic acid sequence encoding proteins that act as transcriptional repressors, antisense molecules, ribozymes, small inhibitory nucleic acid sequences, for example but are not limited to RNAi, shRNAi, siRNA, micro RNAi (mRNAi), antisense oligonucleotides, etc. In general, RNA interference technology is well known in the art, as are methods of delivering RNA interfering agents. See, e.g., U.S. Patent Pub. No. 2010/0221226.

As used herein, gene silencing or gene silenced in reference to an activity of a RNAi molecule, for example a siRNA or miRNA refers to a decrease in the mRNA level in a cell for a target gene by at least about 5%, about 10%, about 20%, about 30%, about 40%, about 50%, about 60%, about 70%, about 80%, about 90%, about 95%, about 99%, about 100% of the mRNA level found in the cell without the presence of the miRNA or RNA interference molecule. In one preferred embodiment, the mRNA levels are decreased by at least about 70%, about 80%, about 90%, about 95%, about 99%, about 100%.

As used herein, the term “RNAi” refers to any type of interfering RNA, including but are not limited to, siRNAi, shRNAi, endogenous microRNA and artificial microRNA. For instance, it includes sequences previously identified as siRNA, regardless of the mechanism of down-stream processing of the RNA (i.e. although siRNAs are believed to have a specific method of in vivo processing resulting in the cleavage of mRNA, such sequences can be incorporated into the vectors in the context of the flanking sequences described herein.

As used herein an “siRNA” refers to a nucleic acid that forms a double stranded RNA, which double stranded RNA has the ability to reduce or inhibit expression of a gene or target gene when the siRNA is present or expressed in the same cell as the target gene, for example where a target gene is Lin28A-recruited TUTase (e.g., Zcchc11 or Zcchc6). The double stranded RNA siRNA can be formed by the complementary strands. In one embodiment, a siRNA refers to a nucleic acid that can form a double stranded siRNA. The sequence of the siRNA can correspond to the full length target gene, or a subsequence thereof. Typically, the siRNA is at least about 15-50 nucleotides in length (e.g., each complementary sequence of the double stranded siRNA is about 15-50 nucleotides in length, and the double stranded siRNA is about 15-50 base pairs in length, preferably about 19-30 base nucleotides, preferably about 20-25 nucleotides in length, e.g., 20, 21, 22, 23, 24, 25, 26, 27, 28, 29, or 30 nucleotides in length.

As used herein “shRNA” or “small hairpin RNA” (also called stem loop) is a type of siRNA. In one embodiment, these shRNAs are composed of a short, e.g. about 19 to about 25 nucleotide, antisense strand, followed by a nucleotide loop of about 5 to about 9 nucleotides, and the analogous sense strand. Alternatively, the sense strand can precede the nucleotide loop structure and the antisense strand can follow.

A stem-loop structure refers to a nucleic acid having a secondary structure that includes a region of nucleotides which are known or predicted to form a double strand (stem portion) that is linked on one side by a region of predominantly single-stranded nucleotides (loop portion). The terms “hairpin” and “fold-back” structures are also used herein to refer to stem-loop structures. Such structures are well known in the art and the term is used consistently with its known meaning in the art. The actual primary sequence of nucleotides within the stem-loop structure is not critical to the practice of the disclosure as long as the secondary structure is present. As is known in the art, the secondary structure does not require exact base-pairing. Thus, the stem may include one or more base mismatches. Alternatively, the base-pairing may be exact, i.e. not include any mismatches. In some instances the precursor microRNA molecule may include more than one stem-loop structure. The multiple stem-loop structures may be linked to one another through a linker, such as, for example, a nucleic acid linker or by a microRNA flanking sequence or other molecule or some combination thereof. The actual primary sequence of nucleotides within the stem-loop structure is not critical as long as the secondary structure is present. As is known in the art, the secondary structure does not require exact base-pairing. Thus, the stem may include one or more base mismatches. Alternatively, the base pairing may not include any mismatches.

CRISPR (Clustered Regularly Interspaced Short Palindromic Repeats) and CRISPR-associated (Cas9) nuclease based gene editing technology for use in disrupting mammalian genes has been extensively described in the art. To target the Cas9 nuclease to the gene to be disrupted, a “single-guide RNA (sgRNA)” or a “guide RNA (gRNA)” may be used to guide the Cas9 nuclease to the target gene. In some embodiments, the TUTase inhibitor of the present disclosure may be a vector that co-expresses an active Cas9 nuclease and an sgRNA that targets the Cas9 nuclease to the gene to be cleaved, e.g., ZCCHC6 or ZCCHC11 gene.

In some embodiments, the TUTase inhibitor can be, for example, an antibody (polyclonal or monoclonal), neutralizing antibody, antibody portion, fragment, analog, variant or derivative, peptide, protein, peptide-mimetic, aptamer, oligonucleotide, hormone, small molecule, nucleic acid, nucleic acid analogue, carbohydrate, or analog, derivative or variant thereof, that function to inactivate the nucleic acid and/or protein of the gene product(s) identified herein, and those as yet unidentified. A protein and/or peptide inhibitor or portion thereof, can be, for example, a mutated protein, therapeutic protein and recombinant protein. Protein and peptide inhibitors can also include for example: mutated protein, genetically modified protein, peptide, synthetic peptide, recombinant protein, chimeric protein, antibody, humanized protein, humanized antibody, chimeric antibody, modified protein and fragment(s) thereof. In some embodiments, the TUTase inhibitor is an anti-ZCCHC6 or anti-ZCCHC11 antibody or an antibody fragment thereof. The antibody fragment used herein has at least the fragment required for antigen binding. Antibody fragments are known to those of ordinary skill in the art.

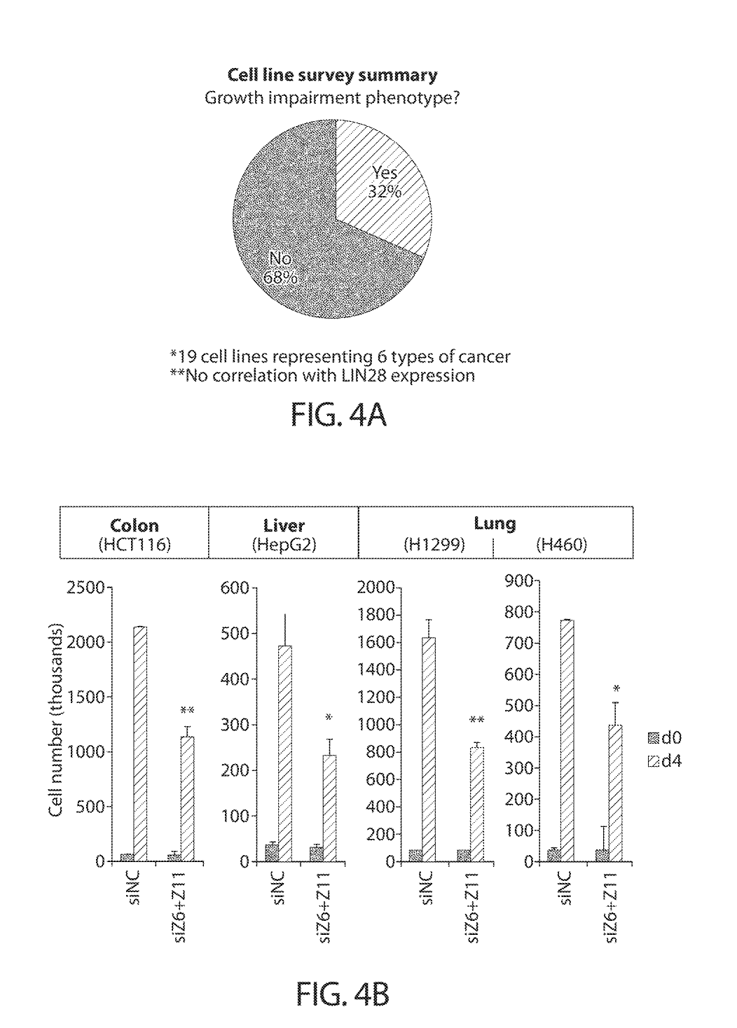

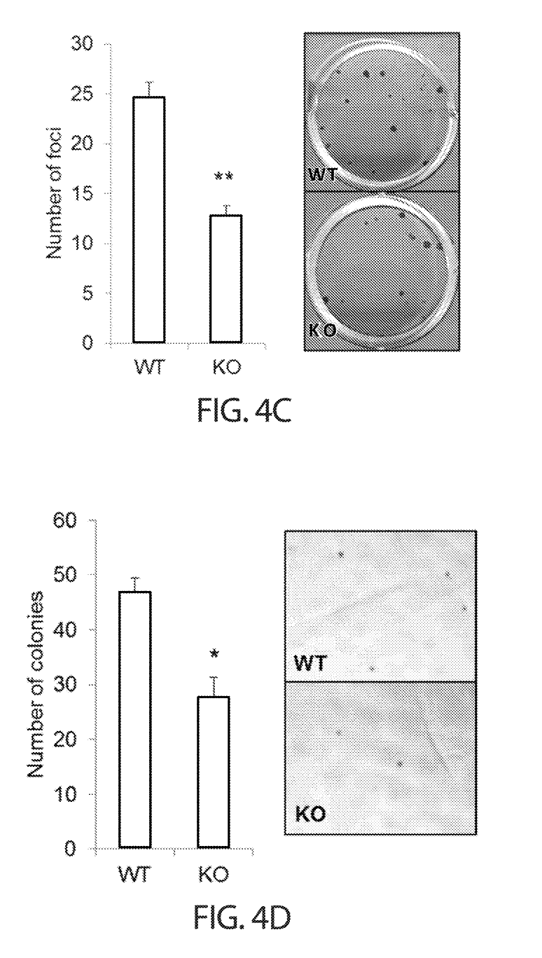

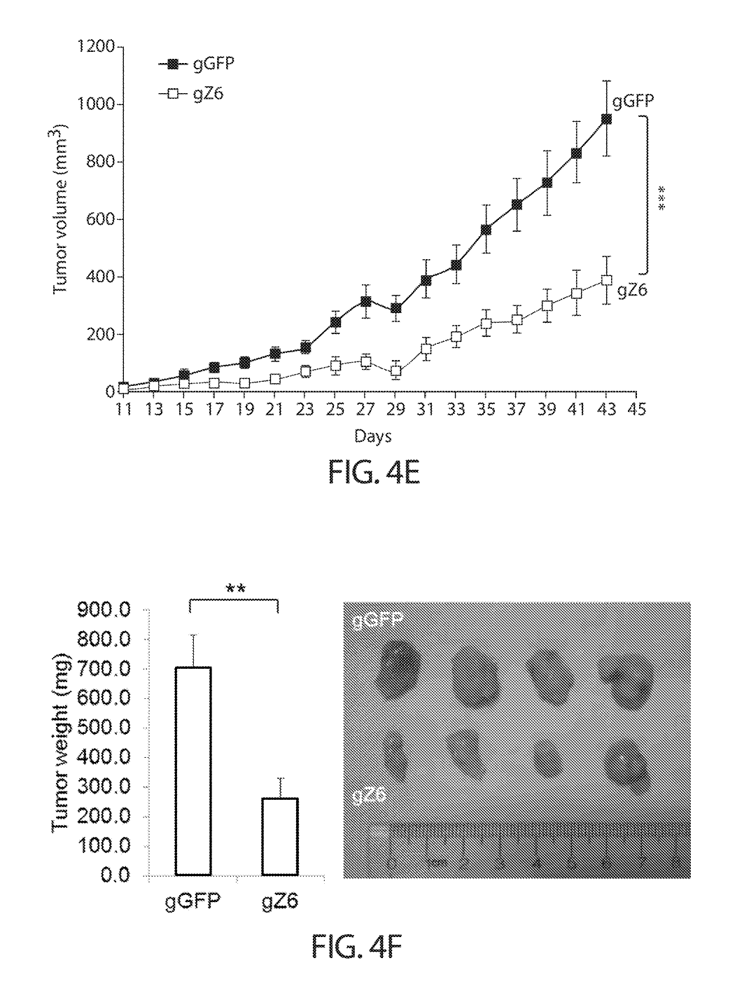

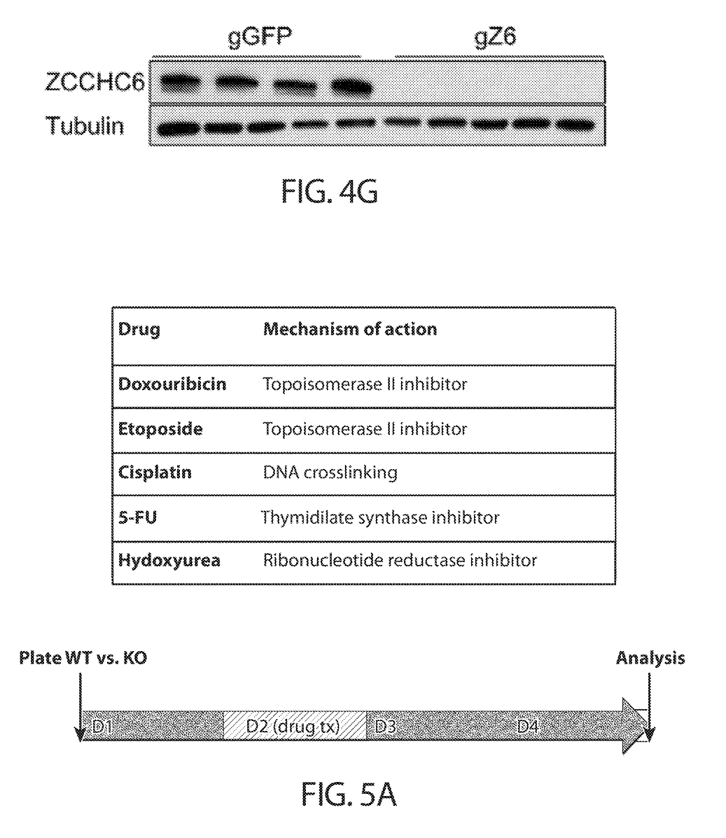

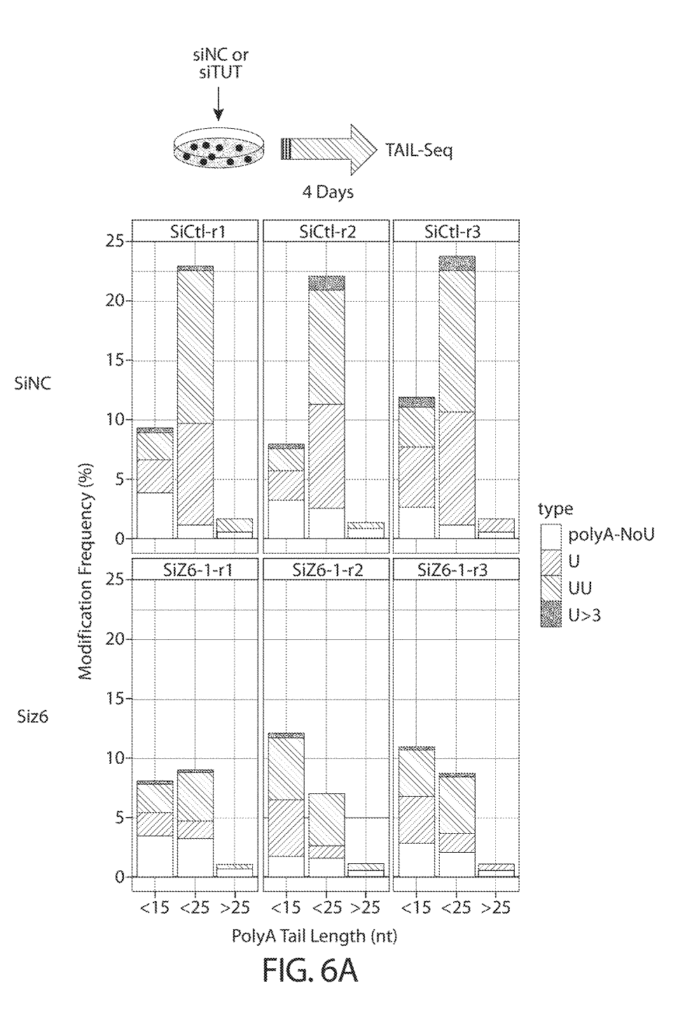

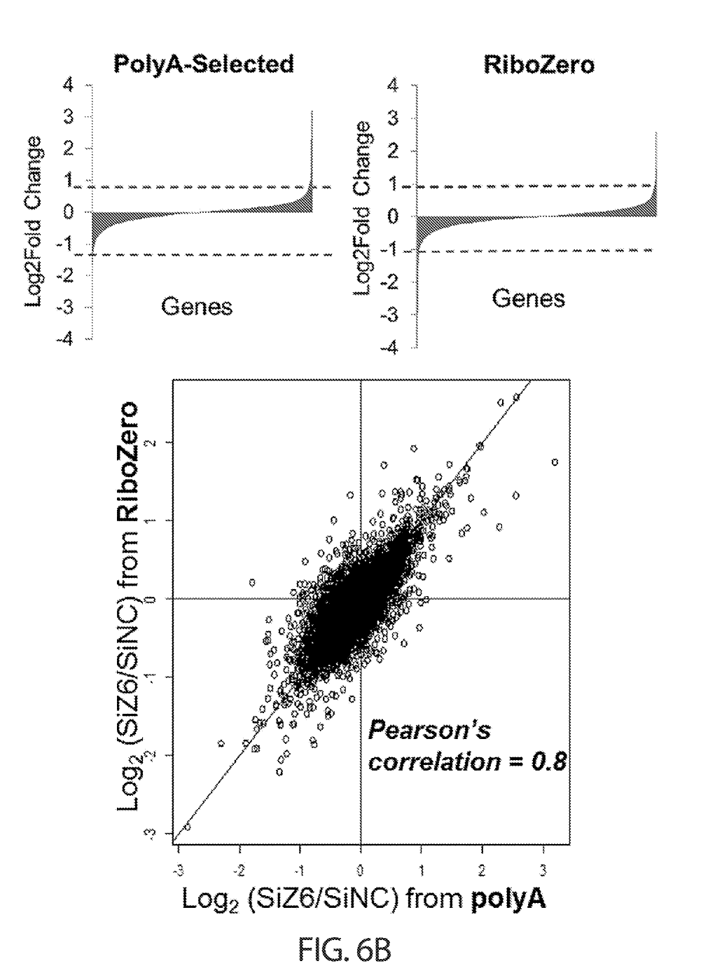

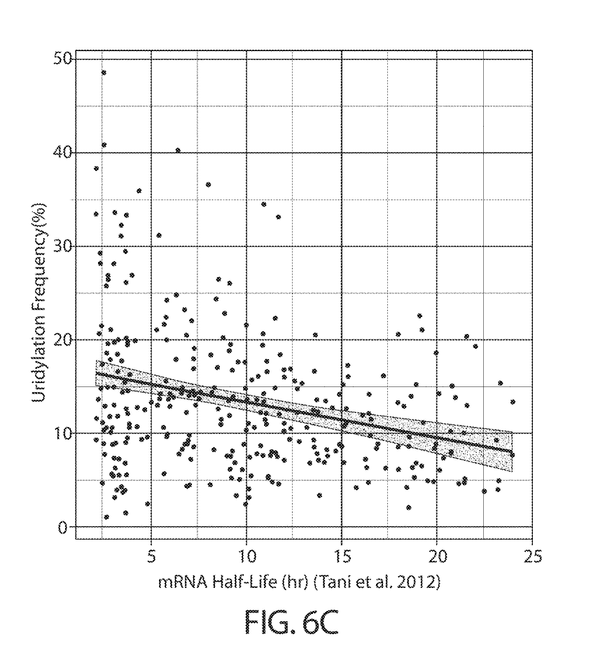

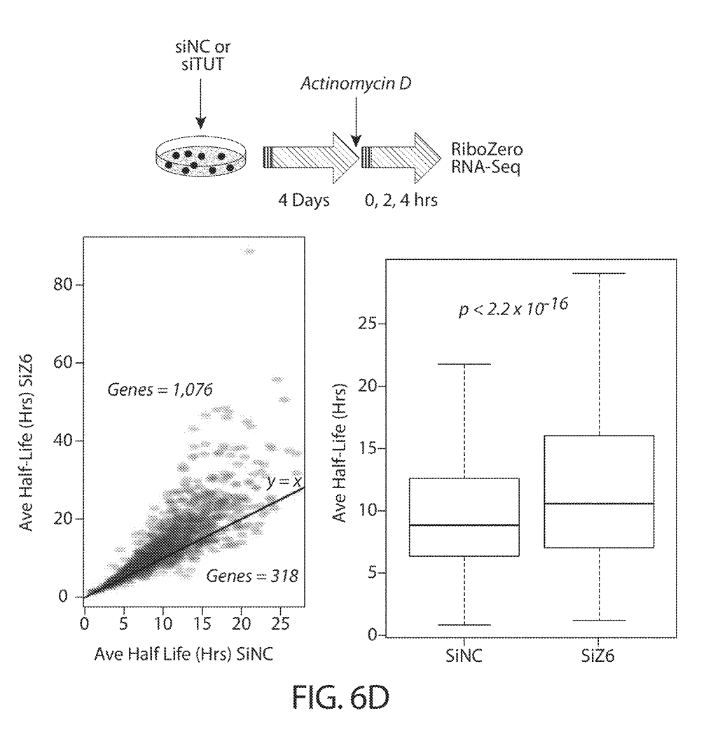

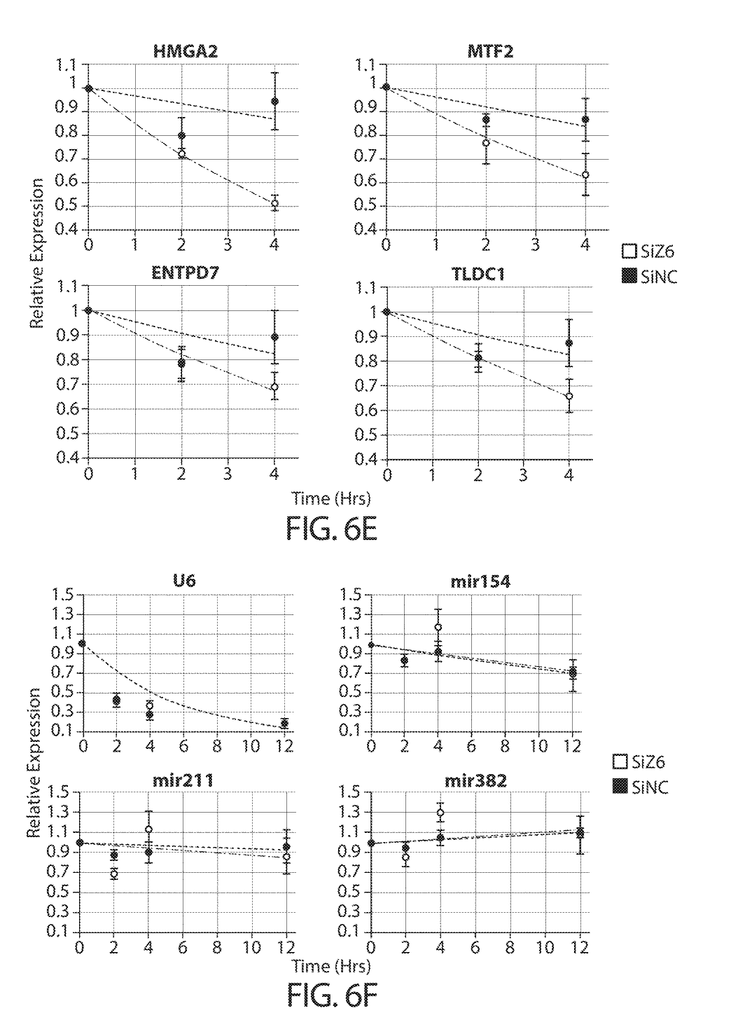

The TUTase inhibitors of the present disclosure, may prevent the uridylation of mRNA in cancer cell and in turn inhibit their turnover. For example, as demonstrated in FIGS. 6A-6F of the present disclosure, uridylation of mRNAs enhance their turn over in cancer cells. FIG. 6D shows that siRNA depletion of ZCCHC6 led to a genome-wide increase of mRNA half-life compared to cells expressing ZCCHC6. Further provided herein are data showing that depletion of TUTase (e.g., ZCCHC6 and/or ZCCHC11) impairs growth in diverse cancer cell types (FIGS. 4A-4G). The term “mRNA half-life,” refers to the amount time it takes for 50% a particular mRNA population to be degraded. mRNA half-life provides information about the stability of different types of mRNAs. Typically, a longer half-life indicates that a certain mRNA is more stable. 3′ uridylation enhances the degradation of mRNAs and thus decreases the half-life. Conversely, when TUTase activity is inhibited and uridylation is absent, mRNA half-life increases.

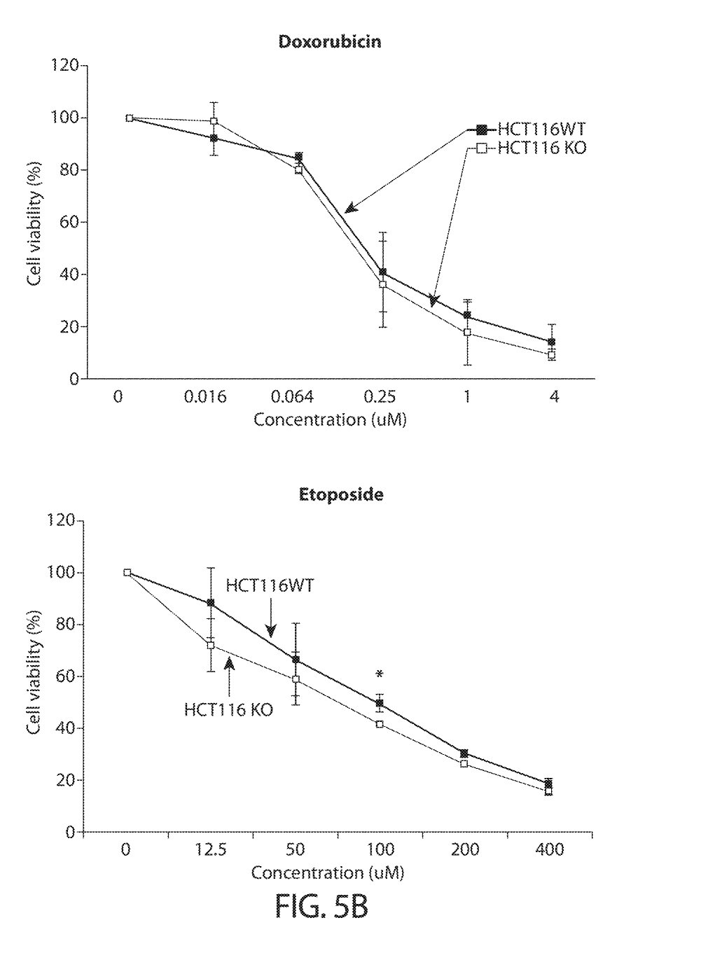

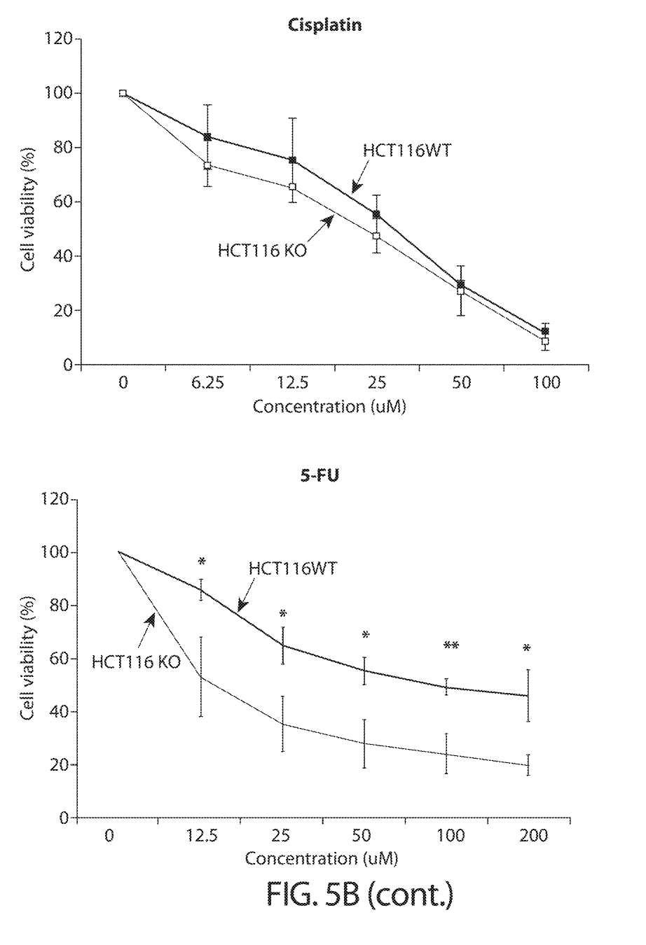

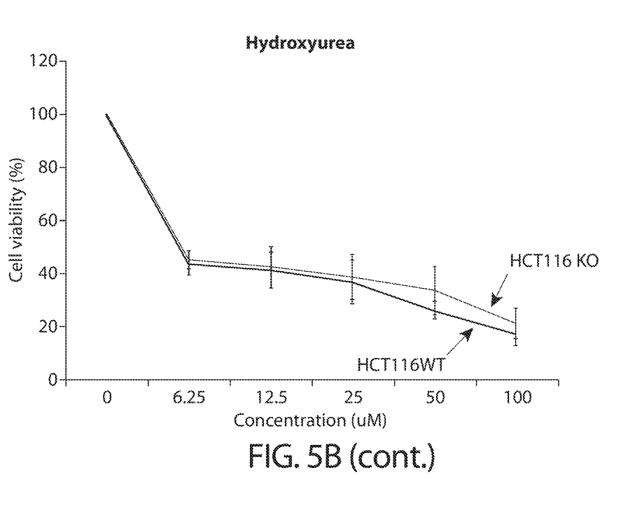

Since TUTase activity is associated with mRNA turnover, they may play an important role in the context of perturbed RNA metabolism. “RNA metabolism,” as used herein, refers to any event in the life cycle of ribonucleic acid (RNA) molecules, including their synthesis, folding/unfolding, modification, processing and degradation. Disruption to any of these process may lead to disruption of the RNA metabolism cycle. For example, inhibition of nucleoside synthesis would result in disruption in RNA synthesis, by making unavailable the building blocks of a RNA molecule. Small molecule agents that are known to inhibit nucleoside synthesis and thereby limit the pool of available nucleotides in the cell (5-fluorouracil and hydroxyurea) are shown in the present disclosure to have a significantly stronger inhibitory effect on cells depleted of TUTase (e.g., ZCCHC6) than corresponding wild-type cells (FIGS. 5A-5B), indicating that cells are sensitized to agents that disrupt RNA metabolism when TUTases are depleted.

Accordingly, the pharmaceutical composition of the present disclosure for treating cancer, may further comprise a therapeutically effective amount of an agent that disrupts RNA metabolism. Agents that disrupt RNA metabolism may be agents that cause DNA/RNA damage (e.g., doxorubicin, cisplatin, etoposide) or agents that inhibit nucleotide synthesis and thereby limit the pool of available nucleotides in the cell (e.g., 5-FU and hydroxyurea). In some embodiments, the agent that disrupts RNA metabolism is an agent that inhibits nucleotide synthesis and metabolism. In some embodiments, the agent that disrupts RNA metabolism is a purine and pyrimidine antimetabolite. In some embodiments, the purine and pyrimidine antimetabolite is a 5′ fluoropyrimidine. In some embodiments, the 5′ fluoropyrimidine is 5-fluorouracil (5-FU), Ftorafur, or uracil tegafur (UFT). In some embodiments, the 5′ fluoropyrimidine is 5-FU. Other purine and pyrimidine antimetabolites that may be used in accordance with the present disclosure include, without limitation, 6-Mercaptopurine, Azathioprine, Fludarabine, Decitabine, Nelarabine, Clofarabine, Vidaza, Capecitabine, Gemcitabine, Pentostatin, Floxuridine, Cytarabine, and 6-thioguanine.

In some embodiments, the agent that disrupts RNA metabolism is 5-FU. 5-FU has been widely used in the treatment of cancer as a chemotherapy agent for decades. 5-FU belongs to a class of drugs termed “antimetabolites,” functioning by inhibiting essential biosynthetic processes in cancer cells, or by incorporated into macromolecules, such as DNA and RNA, and inhibiting their normal function. It is worth noting that not all agents that inhibit nucleotide synthesis have the same strong effect as 5-FU in reducing cancer growth, when used in combination with TUTase depletion. For example, as shown in FIGS. 5A-5B, another agent that inhibits nucleotide synthesis, hydroxyurea, did not further reduce cancer cell grow when used in combination with TUTase depletion, compared to cells depleted of TUTase alone. This may suggest that different agents that disrupt nucleoside synthesis have different mechanisms of action.

In some embodiments, the agent that disrupts RNA metabolism is an antifolate. An “antifolate,” as used herein, refers to a drug that antagonize (i.e., block) the actions of folic acid (vitamin B9). Folic acid's primary function in the body is as a cofactor to various methyltransferases involved in serine, methionine, thymidine and purine biosynthesis. Consequently, antifolates inhibit cell division, DNA/RNA synthesis and repair and protein synthesis. Suitable antifolates that may be used in accordance with the present disclosure include, without limitation, Methotrexate, Pemetrexed, Nolatrexed, Raltitrexed, and ZD9331.

The perturbation of RNA metabolism induced by TUTase depletion, may further lead to protein metabolism perturbation. The term “protein metabolism,” as used herein, refers to the various biochemical processes responsible for the synthesis of proteins and amino acids, and the breakdown of proteins (and other large molecules) by catabolism. In some embodiments, the disruption in protein metabolism is a disruption in protein turnover. The term “protein turnover,” refers to the balance between protein synthesis and protein degradation. More synthesis than breakdown indicates an anabolic state that builds lean tissues, more breakdown than synthesis indicates a catabolic state that burns lean tissues. Thus, perturbations in protein synthesis rate and/or protein degradation rate may disrupt protein turnover.

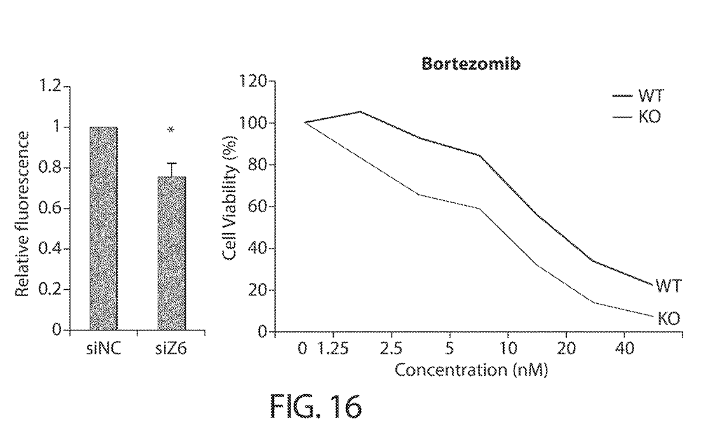

Provided herein are data showing that TUTase depletion led to a 30% reduction of translation rate, and that TUTase depleted cells are sensitized to disruption of protein turnover (FIG. 16). Thus, the pharmaceutical composition of the present disclosure for treating cancer, may further comprise an agent that disrupts protein metabolism. In some embodiments, the agent that disrupts protein metabolism inhibits protein turnover.

Proteasomes are protein complexes inside all eukaryotes and archaea, and in some bacteria. The main function of the proteasome is to degrade unneeded or damaged proteins by proteolysis, a chemical reaction that breaks peptide bonds. In eukaryotes, proteasomes are located in the nucleus and the cytoplasm. The proteasome is the most important machinery involved in protein degradation in eukaryotic cells. Inhibition of the proteasome effectively inhibits protein degradation and then protein turnover. Thus, the agent that disrupts protein turnover of the present disclosure, may be a proteasome inhibitor. A number of proteasome inhibitors have been described in the art and have been used in cancer treatment, due to the stress the cell endures when the proteasome is rendered non-functional. The proteasome inhibitors that may be used in accordance with the present disclosure include, without limitation, bortezomib, Lxazomib, Carfilzomib, Oprozomib (ONX-0912), Delanzomib (CEP-18770), Marizomib (salinosporamide A), Lactacystin, Disulfiram Epigallocatechin-3-gallate, Epoxomicin, MG132Beta-hydroxy beta-methylbutyrate, and combinations thereof. In some embodiments, the proteasome inhibitor is bortezomib.

In some embodiments, the agent that disrupts protein metabolism is an agent that inhibits the PIK3/mTOR pathway. The mTOR protein is a 289-kDa serine-threonine kinase that belongs to the phospho-inositide 3-kinase (PI3K)-related kinase family and is conserved throughout evolution. mTOR nucleates at least two distinct multi-protein complexes, mTOR complex 1 (mTORC1) and mTOR complex 2 (mTORC2) (reviewed by Guertin and Sabatini, 2007). mTORC1 positively controls protein synthesis, which is required for cell growth, through various downstream effectors. mTORC1 promotes protein synthesis by phosphorylating the eukaryotic initiation factor 4E (eIF4E)-binding protein 1 (4E-BP1) and the p70 ribosomal S6 kinase 1 (S6K1). The phosphorylation of 4E-BP1 prevents its binding to eIF4E, enabling eIF4E to promote cap-dependent translation (reviewed by Richter and Sonenberg, 2005). The stimulation of S6K1 activity by mTORC1 leads to increases in mRNA biogenesis, cap-dependent translation and elongation, and the translation of ribosomal proteins through regulation of the activity of many proteins, such as S6K1 aly/REF-like target (SKAR), programmed cell death 4 (PDCD4), eukaryotic elongation factor 2 kinase (eEF2K) and ribosomal protein S6 (reviewed by Ma and Blenis, 2009). The activation of mTORC1 has also been shown to promote ribosome biogenesis by stimulating the transcription of ribosomal RNA through a process involving the protein phosphatase 2A (PP2A) and the transcription initiation factor IA (TIF-IA) (Mayer et al., 2004). In contrast, inhibition of mTORC1 inhibits protein synthesis and disrupts protein metabolism.

Thus, the agent that disrupts protein metabolism of the present disclosure, may be a PIK3/mTOR inhibitor. In some embodiments, the PIK3/mTOR inhibitor is rapamycin or a rapalog. Suitable rapalogs that may be used in accordance with the present disclosure include, without limitation, Sirolimus, Temsirolimus, Everolimus, Deforolimus, and combinations thereof. In some embodiments, the PIK3/mTOR inhibitor is an ATP-competitive mTOR kinase inhibitor. In some embodiments, the ATP-competitive mTOR kinase inhibitor is Torin1 or Torin2. Any PIK3/mTOR inhibitor may be used in accordance with the present disclosure.

It is to be understood that the pharmaceutical composition of the present disclosure encompasses compositions comprising a TUTase inhibitor and an agent that disrupts RNA metabolism, compositions comprising a TUTase inhibitor and an agent that disrupts protein metabolism, and compositions comprising a TUTase inhibitor, an agent that disrupts RNA metabolism, and an agent that disrupts protein metabolism.

The pharmaceutical composition of the present disclosure, may further comprise a pharmaceutically acceptable carrier. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier” means a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. Each carrier must be “acceptable” in the sense of being compatible with the other ingredients of the formulation.

The methods and pharmaceutical compositions of the present disclosure are used to treat cancer in a subject in need thereof. The types of cancer that may be treated using the methods disclosed herein include, without limitation neoplasms, malignant tumors, metastases, or any disease or disorder characterized by uncontrolled cell growth such that it would be considered cancerous. The cancer may be a primary or metastatic cancer. Cancers include, but are not limited to, biliary tract cancer; bladder cancer; brain cancer including glioblastoma and medulloblastoma; breast cancer; cervical cancer; choriocarcinoma; colon cancer; endometrial cancer; esophageal cancer; gastric cancer; hematological neoplasms including acute lymphocytic and myelogenous leukemia; multiple myeloma; AIDS-associated leukemia and adult T-cell leukemia lymphoma; intraepithelial neoplasm including Bowen's disease and Paget's disease; liver cancer; lung cancer; lymphomas including Hodgkin's disease and lymphocytic lymphoma; neuroblastoma; oral cancer including squamous cell carcinoma; ovarian cancer including those arising from epithelial cells, stromal cells, germ cells and mesenchymal cells; pancreatic cancer; prostate cancer; rectal cancer; sarcomas including leiomyosarcoma, rhabdomyosarcoma, liposarcoma, fibrosarcoma, and osteosarcoma; skin cancer including melanoma, Kaposi's sarcoma, basocellular cancer, and squamous cell cancer; testicular cancer including germinal tumors such as seminoma, non-seminoma, teratomas, choriocarcinoma; stromal tumor and germ cell tumor; thyroid cancer including thyroid adenocarcinoma and medullar carcinoma; and renal cancer including adenocarcinoma and Wilms' tumor. Commonly encountered cancers include breast, prostate, lung, ovarian, colorectal, and brain cancer. In some embodiments, the cancer cells are metastatic. In some embodiments, the cancer does not express LIN28A/B.

In its broadest sense, the terms “treatment” or “to treat” refer to both therapeutic and prophylactic treatments. If the subject in need of treatment is has cancer, then “treating the condition” refers to ameliorating, reducing or eliminating one or more symptoms associated with the cancer or the severity of cancer or preventing any further progression of cancer. If the subject in need of treatment is one who is at risk of having cancer, then treating the subject refers to reducing the risk of the subject having cancer or preventing the subject from developing cancer.

A subject shall mean a human or vertebrate animal or mammal including but not limited to a rodent, e.g., a rat or a mouse, dog, cat, horse, cow, pig, sheep, goat, turkey, chicken, and primate, e.g., monkey. The methods of the present disclosure are useful for treating a subject in need thereof. A subject in need thereof can be a subject who has a risk of developing cancer (i.e., via a genetic test) or a subject who has cancer.

Therapeutic compounds or agents, e.g., TUTase inhibitors and/or agents that disrupt protein/RNA metabolism, that may be used in accordance with the present disclosure may be directly administered to the subject or may be administered in conjunction with a delivery device or vehicle. Delivery vehicles or delivery devices for delivering therapeutic compounds to surfaces have been described. The therapeutic compounds of the present disclosure may be administered alone (e.g., in saline or buffer) or using any delivery vehicle(s) known in the art.

The term “therapeutically effective amount” of the present disclosure refers to the amount necessary or sufficient to realize a desired biologic effect. For example, a therapeutically effective amount of a TUTase inhibitor associated with the present disclosure may be that amount sufficient to ameliorate one or more symptoms of cancer. Combined with the teachings provided herein, by choosing among the various active compounds and weighing factors such as potency, relative bioavailability, patient body weight, severity of adverse side-effects and preferred mode of administration, an effective prophylactic or therapeutic treatment regimen can be planned which does not cause substantial toxicity and yet is entirely effective to treat the particular subject. The effective amount for any particular application can vary depending on such factors as the disease or condition being treated, the particular therapeutic compounds being administered the size of the subject, or the severity of the disease or condition. One of ordinary skill in the art can empirically determine the effective amount of a particular therapeutic compound associated with the present disclosure without necessitating undue experimentation.

In some embodiments, the agents and pharmaceutical compositions as disclosed herein comprising at least one inhibitor of TUTase can be administered in therapeutically effective dosages to provide a beneficial effect, e.g. reducing tumor size, slowing rate of tumor growth, reducing cell proliferation of the tumor, promoting cancer cell death, inhibiting angiogenesis, inhibiting metastasis, or otherwise improving overall clinical condition, without necessarily eradicating the cancer.

The term “reduce tumor size,” as used herein, refers to the decrease in tumor size compared to before the subject was treated using the methods and the compositions of the present disclosure. In some embodiments, the tumor size is reduced by at least 10%, at least 20%, at least 30%, at least 50%, at least 60%, at least 70%, at least 80%, at least 90%, at least 99%. In some embodiments, the tumor size is reduced by 100%, i.e., the tumor disappears. In some embodiments, the tumor is reduced to no more that 80%, no more than 70%, no more than 60%, no more than 40%, no more than 30%, no more than 20%, no more than 10% no more than 5%, no more than 1%, or no more than 0.1% of its original size.

In some embodiments, the compositions and methods of the present disclosure, when administered to the subject, prevents metastasis of the cancer. The term “metastasis” refers to the spread of a primary tumor from one organ or part of the body to another not directly connected with it. A “primary tumor” refers to a tumor growing at the anatomical site where tumor progression began and proceeded to yield a cancerous mass. Most cancers develop at their primary site but then go on to spread to other parts of the body, i.e., metastasis. These further tumors are secondary tumors. Metastasis results from several interconnected processes including cell proliferation, angiogenesis, cell adhesion, migration, and invasion into the surrounding tissue. The term “prevent metastasis” means the process of a primary to spread to other parts of the body that is not directly connected is inhibited, or that the development of the secondary tumor is prevented.

Subject doses of the compounds described herein for delivery typically range from about 0.1 μg to 10 mg per administration, which depending on the application could be given daily, weekly, or monthly and any other amount of time there between. In some embodiments a single dose is administered during the critical consolidation or reconsolidation period. The doses for these purposes may range from about 10 μg to 5 mg per administration, and most typically from about 100 μg to 1 mg, with 2-4 administrations being spaced, for example, days or weeks apart, or more. In some embodiments, however, parenteral doses for these purposes may be used in a range of 5 to 10,000 times higher than the typical doses described above.

In some embodiments a compound of the present disclosure is administered at a dosage of between about 1 and 10 mg/kg of body weight of the mammal. In other embodiments a compound of the present disclosure is administered at a dosage of between about 0.001 and 1 mg/kg of body weight of the mammal. In yet other embodiments a compound of the present disclosure is administered at a dosage of between about 10-100 ng/kg, 100-500 ng/kg, 500 ng/kg-1 mg/kg, or 1-5 mg/kg of body weight of the mammal, or any individual dosage therein.

The formulations of the present disclosure are administered in pharmaceutically acceptable solutions, which may routinely contain pharmaceutically acceptable concentrations of salt, buffering agents, preservatives, compatible carriers, and optionally other therapeutic ingredients.

For use in therapy, an effective amount of the therapeutic compound associated with the present disclosure can be administered to a subject by any mode that delivers the therapeutic agent or compound to the desired surface, e.g., mucosal, injection to cancer, systemic, etc. Administering the pharmaceutical composition of the present disclosure may be accomplished by any means known to the skilled artisan. Preferred routes of administration include but are not limited to oral, parenteral, intravenous, intramuscular, intranasal, sublingual, intratracheal, inhalation, ocular, vaginal, rectal and intracerebroventricular.

For oral administration, the therapeutic compounds of the present disclosure can be formulated readily by combining the active compound(s) with pharmaceutically acceptable carriers well known in the art. Such carriers enable the compounds of the present disclosure to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl cellulose, sodium carboxymethylcellulose, and/or polyvinylpyrrolidone (PVP). If desired, disintegrating agents may be added, such as the cross linked polyvinyl pyrrolidone, agar, or alginic acid or a salt thereof such as sodium alginate. Optionally the oral formulations may also be formulated in saline or buffers, i.e., EDTA for neutralizing internal acid conditions or may be administered without any carriers.

Also specifically contemplated are oral dosage forms of the above component or components. The component or components may be chemically modified so that oral delivery of the derivative is efficacious. Generally, the chemical modification contemplated is the attachment of at least one moiety to the component molecule itself, where said moiety permits (a) inhibition of proteolysis; and (b) uptake into the blood stream from the stomach or intestine. Also desired is the increase in overall stability of the component or components and increase in circulation time in the body. Examples of such moieties include: polyethylene glycol, copolymers of ethylene glycol and propylene glycol, carboxymethyl cellulose, dextran, polyvinyl alcohol, polyvinyl pyrrolidone and polyproline (Abuchowski and Davis, 1981, “Soluble Polymer-Enzyme Adducts” In: Enzymes as Drugs, Hocenberg and Roberts, eds., Wiley-Interscience, New York, N.Y., pp. 367-383; Newmark, et al., 1982, J. Appl. Biochem. 4:185-189). Other polymers that could be used are poly-1,3-dioxolane and poly-1,3,6-tioxocane. Preferred for pharmaceutical usage, as indicated above, are polyethylene glycol moieties.

The location of release may be the stomach, the small intestine (the duodenum, the jejunum, or the ileum), or the large intestine. One skilled in the art has available formulations which will not dissolve in the stomach, yet will release the material in the duodenum or elsewhere in the intestine. Preferably, the release will avoid the deleterious effects of the stomach environment, either by protection of the therapeutic agent or by release of the biologically active material beyond the stomach environment, such as in the intestine.

To ensure full gastric resistance a coating impermeable to at least pH 5.0 is preferred. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac. These coatings may be used as mixed films.

A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic i.e., powder; for liquid forms, a soft gelatin shell may be used. The shell material of cachets could be thick starch or other edible paper. For pills, lozenges, molded tablets or tablet triturates, moist massing techniques can be used.

The therapeutic can be included in the formulation as fine multi particulates in the form of granules or pellets of particle size about 1 mm. The formulation of the material for capsule administration could also be as a powder, lightly compressed plugs or even as tablets. The therapeutic could be prepared by compression.

Colorants and flavoring agents may all be included. For example, the therapeutic agent may be formulated (such as by liposome or microsphere encapsulation) and then further contained within an edible product, such as a refrigerated beverage containing colorants and flavoring agents.

One may dilute or increase the volume of the therapeutic with an inert material. These diluents could include carbohydrates, especially mannitol, a lactose, anhydrous lactose, cellulose, sucrose, modified dextrans and starch. Certain inorganic salts may be also be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of the therapeutic into a solid dosage form. Materials used as disintegrates include but are not limited to starch, including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrants are the insoluble cationic exchange resins. Powdered gums may be used as disintegrants and as binders and these can include powdered gums such as agar, Karaya or tragacanth. Alginic acid and its sodium salt are also useful as disintegrants.

Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic.

An anti-frictional agent may be included in the formulation of the therapeutic to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 and 6000.

Glidants that might improve the flow properties of the drug during formulation and to aid rearrangement during compression might be added. The glidants may include starch, talc, pyrogenic silica and hydrated silicoaluminate.

To aid dissolution of the therapeutic into the aqueous environment a surfactant might be added as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethomium chloride. The list of potential nonionic detergents that could be included in the formulation as surfactants are lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the therapeutic agent either alone or as a mixture in different ratios.

Pharmaceutical preparations which can be used orally include push fit capsules made of gelatin, as well as soft, sealed capsules made of gelatin and a plasticizer, such as glycerol or sorbitol. The push fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For administration by inhalation, the compounds for use according to the present disclosure may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

Also contemplated herein is pulmonary delivery of the therapeutic compounds of the present disclosure. The therapeutic agent is delivered to the lungs of a mammal while inhaling and traverses across the lung epithelial lining to the blood stream. Other reports of inhaled molecules include Adjei et al., 1990, Pharmaceutical Research, 7:565 569; Adjei et al., 1990, International Journal of Pharmaceutics, 63:135 144 (leuprolide acetate); Braquet et al., 1989, Journal of Cardiovascular Pharmacology, 13(suppl. 5):143 146 (endothelin-1); Hubbard et al., 1989, Annals of Internal Medicine, Vol. III, pp. 206 212 (al antitrypsin); Smith et al., 1989, J. Clin. Invest. 84:1145-1146 (a 1-proteinase); Oswein et al., 1990, “Aerosolization of Proteins”, Proceedings of Symposium on Respiratory Drug Delivery II, Keystone, Colo., March, (recombinant human growth hormone); Debs et al., 1988, J. Immunol. 140:3482 3488 (interferon g and tumor necrosis factor alpha) and Platz et al., U.S. Pat. No. 5,284,656 (granulocyte colony stimulating factor). A method and composition for pulmonary delivery of drugs for systemic effect is described in U.S. Pat. No. 5,451,569, issued Sep. 19, 1995 to Wong et al.

Contemplated for use in the practice of this present disclosure are a wide range of mechanical devices designed for pulmonary delivery of therapeutic products, including but not limited to nebulizers, metered dose inhalers, and powder inhalers, all of which are familiar to those skilled in the art.

Some specific examples of commercially available devices suitable for the practice of this present disclosure are the Ultravent nebulizer, manufactured by Mallinckrodt, Inc., St. Louis, Mo.; the Acorn II nebulizer, manufactured by Marquest Medical Products, Englewood, Colo.; the Ventolin metered dose inhaler, manufactured by Glaxo Inc., Research Triangle Park, N.C.; and the Spinhaler powder inhaler, manufactured by Fisons Corp., Bedford, Mass.

All such devices require the use of formulations suitable for the dispensing of therapeutic agent. Typically, each formulation is specific to the type of device employed and may involve the use of an appropriate propellant material, in addition to the usual diluents, and/or carriers useful in therapy. Also, the use of liposomes, microcapsules or microspheres, inclusion complexes, or other types of carriers is contemplated. Chemically modified therapeutic agent may also be prepared in different formulations depending on the type of chemical modification or the type of device employed.

Formulations suitable for use with a nebulizer, either jet or ultrasonic, will typically comprise therapeutic agent dissolved in water at a concentration of about 0.1 to 25 mg of biologically active compound per mL of solution. The formulation may also include a buffer and a simple sugar (e.g., for stabilization and regulation of osmotic pressure). The nebulizer formulation may also contain a surfactant, to reduce or prevent surface induced aggregation of the compound caused by atomization of the solution in forming the aerosol.

Formulations for use with a metered dose inhaler device will generally comprise a finely divided powder containing the therapeutic agent suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2 tetrafluoroethane, or combinations thereof. Suitable surfactants include sorbitan trioleate and soya lecithin. Oleic acid may also be useful as a surfactant.

Formulations for dispensing from a powder inhaler device will comprise a finely divided dry powder containing therapeutic agent and may also include a bulking agent, such as lactose, sorbitol, sucrose, or mannitol in amounts which facilitate dispersal of the powder from the device, e.g., 50 to 90% by weight of the formulation. The therapeutic agent should most advantageously be prepared in particulate form with an average particle size of less than 10 mm (or microns), most preferably 0.5 to 5 mm, for most effective delivery to the distal lung.

Intra-nasal delivery of a pharmaceutical composition of the present disclosure is also contemplated. Intra-nasal delivery allows the passage of a pharmaceutical composition of the present disclosure to the blood stream directly after administering the therapeutic product to the nose, without the necessity for deposition of the product in the lung. Formulations for nasal delivery include those with dextran or cyclodextran.

For nasal administration, a useful device is a small, hard bottle to which a metered dose sprayer is attached. In one embodiment, the metered dose is delivered by drawing the pharmaceutical composition of the present disclosure solution into a chamber of defined volume, which chamber has an aperture dimensioned to aerosolize and aerosol formulation by forming a spray when a liquid in the chamber is compressed. The chamber is compressed to administer the pharmaceutical composition of the present disclosure. In a specific embodiment, the chamber is a piston arrangement. Such devices are commercially available.

Alternatively, a plastic squeeze bottle with an aperture or opening dimensioned to aerosolize an aerosol formulation by forming a spray when squeezed is used. The opening is usually found in the top of the bottle, and the top is generally tapered to partially fit in the nasal passages for efficient administration of the aerosol formulation. Preferably, the nasal inhaler will provide a metered amount of the aerosol formulation, for administration of a measured dose of the drug.

The agents, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active compounds may be in powder form for constitution with a suitable vehicle, e.g., sterile pyrogen-free water, before use.

In addition to the formulations described previously, the compounds may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer, Science 249:1527-1533, 1990, which is incorporated herein by reference.

The therapeutic compounds of the present disclosure and optionally other therapeutics may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

The pharmaceutical compositions of the present disclosure contain an effective amount of a therapeutic compound of the present disclosure optionally included in a pharmaceutically-acceptable carrier. The term pharmaceutically-acceptable carrier means one or more compatible solid or liquid filler, diluents or encapsulating substances which are suitable for administration to a human or other vertebrate animal. The term carrier denotes an organic or inorganic ingredient, natural or synthetic, with which the active ingredient is combined to facilitate the application. The components of the pharmaceutical compositions also are capable of being commingled with the compounds of the present disclosure, and with each other, in a manner such that there is no interaction which would substantially impair the desired pharmaceutical efficiency.

The therapeutic agents may be delivered to the brain using a formulation capable of delivering a therapeutic agent across the blood brain barrier. One obstacle to delivering therapeutics to the brain is the physiology and structure of the brain. The blood-brain barrier is made up of specialized capillaries lined with a single layer of endothelial cells. The region between cells are sealed with a tight junction, so the only access to the brain from the blood is through the endothelial cells. The barrier allows only certain substances, such as lipophilic molecules through and keeps other harmful compounds and pathogens out. Thus, lipophilic carriers are useful for delivering non-lipophilic compounds to the brain. For instance, DHA, a fatty acid naturally occurring in the human brain has been found to be useful for delivering drugs covalently attached thereto to the brain (Such as those described in U.S. Pat. No. 6,407,137). U.S. Pat. No. 5,525,727 describes a dihydropyridine pyridinium salt carrier redox system for the specific and sustained delivery of drug species to the brain. U.S. Pat. No. 5,618,803 describes targeted drug delivery with phosphonate derivatives. U.S. Pat. No. 7,119,074 describes amphiphilic prodrugs of a therapeutic compound conjugated to an PEG-oligomer/polymer for delivering the compound across the blood brain barrier. Others are known to those of skill in the art.

The therapeutic agents of the present disclosure may be delivered with other therapeutics for treating cancer.

Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. The nomenclatures utilized in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

The following examples are provided to illustrate specific instances of the practice of the present disclosure and are not intended to limit the scope of the present disclosure. As will be apparent to one of ordinary skill in the art, the present disclosure will find application in a variety of compositions and methods.

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

### Example 1. Uridylation-Mediated mRNA Turnover Promotes Oncogenesis and Supports Cancer Cell Growth

### Investigation of the Role of the TUTases ZCCHC6 and ZCCHC11 in Oncogenesis: Preliminary Data

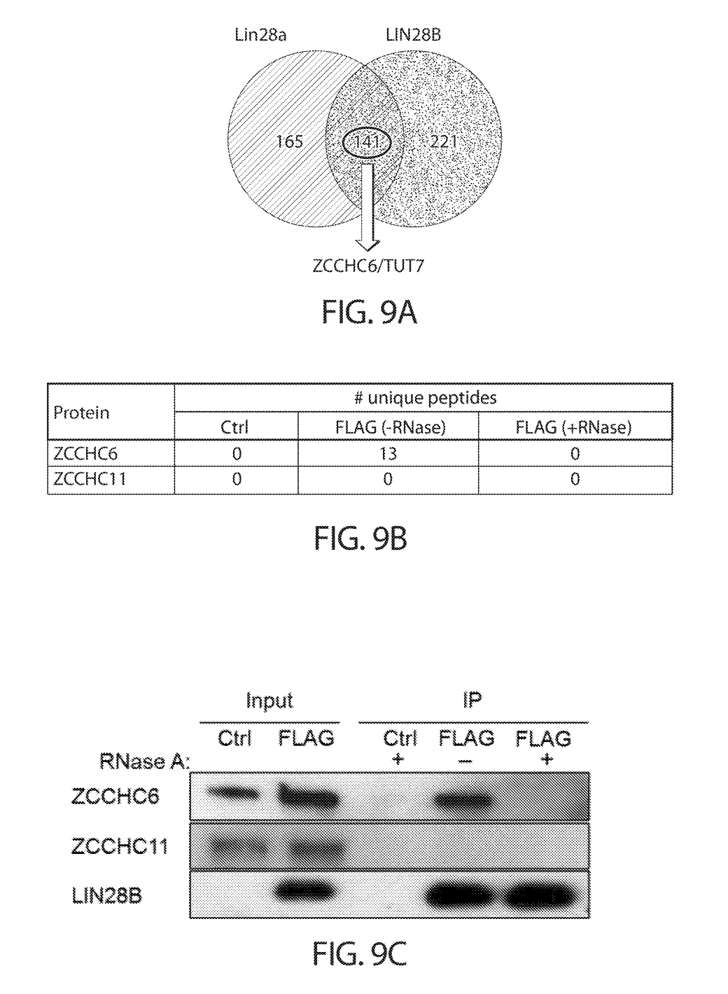

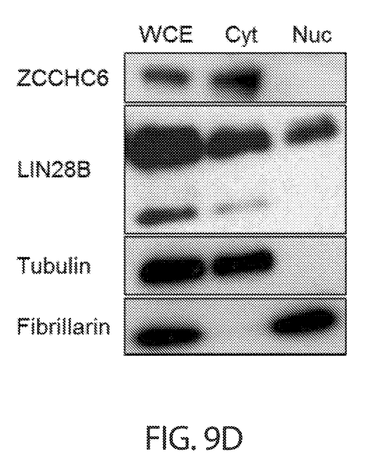

There are two vertebrate LIN28 homologs, LIN28/LIN28A and LIN28B, yet their mechanistic and functional relationship is unclear. According to an originally proposed model, LIN28A and LIN28B have distinct mechanisms of let-7 suppression, whereby LIN28A requires pre-let-7 uridylation via ZCCHC6/TUT7 or an alternative TUTase, ZCCHC11/TUT4, while LIN28B sequesters pri-let-7 in the nucleolus [1-3]. Thus, it was surprising to identify ZCCHC6 as a partner of LIN28B through a protein-protein interaction analysis in neuroblastoma cells (FIGS. 9A-9C). Subcellular fractionation followed by Western blot further indicated that LIN28B and ZCCHC6 co-localize in the cytoplasm, ruling out the possibility of a nucleolar-based interaction (FIG. 9D). Taken together, these results showed that LIN28B and ZCCHC6 interact in vivo and suggested that ZCCHC6 may uridylate pre-let-7 in a LIN28B-mediated manner, which has indeed been confirmed in a recent publication [4].

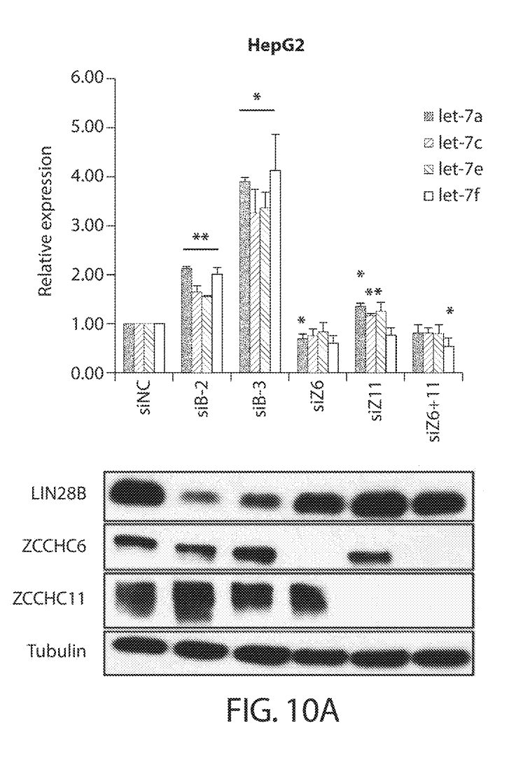

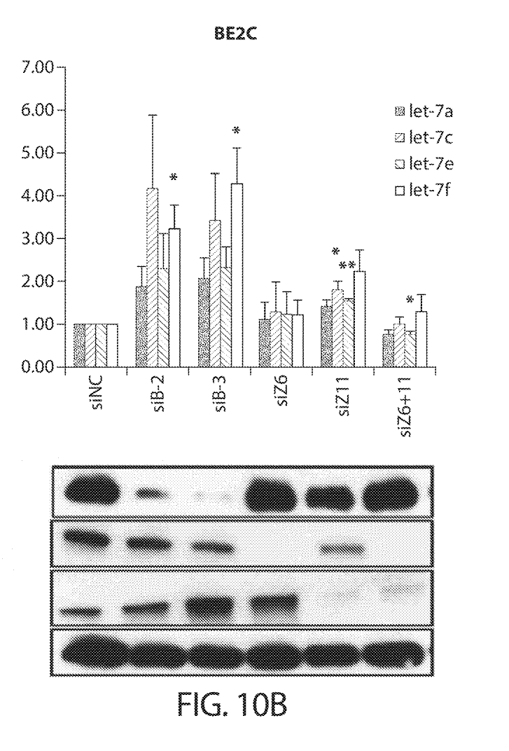

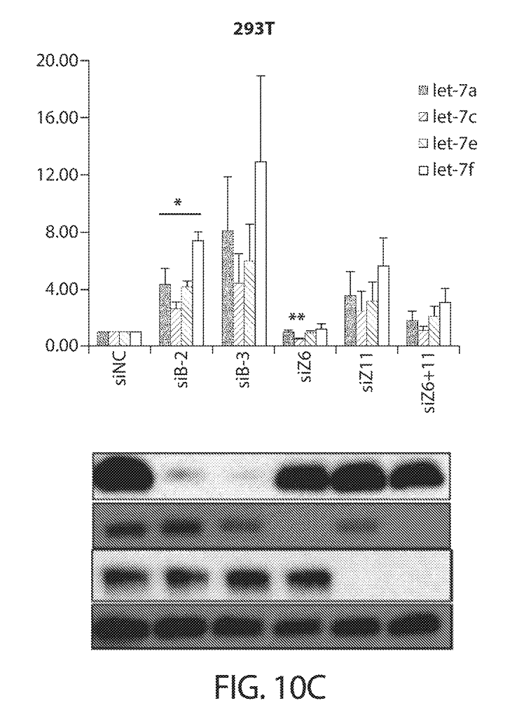

To determine if ZCCHC6 regulates let-7, siRNA knockdowns were performed in three LIN28B-expressing cell lines, including those used in the above experiments. Surprisingly, ZCCHC6 knockdown did not have an effect on mature let-7 levels and even led to a mild reduction in the levels of a few let-7 species, while LIN28B knockdown caused expected let-7 derepression (FIG. 10A-10C). Since ZCCHC11 can act redundantly with ZCCHC6, potentially compensating for its loss [3], ZCCHC11 knockdowns individually or in combination with ZCCHC6 were also performed. While single knockdowns led to a mild increase in the levels of individual let-7s, the response was not consistent across let-7 species or cell types, and double knockdowns had no appreciable effect at all (FIGS. 10A-10C). Taken together, these data indicate that ZCCHC6 and ZCCHC11 do not universally regulate let-7 in conjunction with LIN28A/B.

As the other major targets of LIN28A/B are mRNAs, whether the TUTases regulate mRNAs via non-templated uridylation was examined. A few pioneering studies had reported a role for 3′ uridylation on a small number of mRNA targets—mostly in regulating their decay [5-7]—so it was surmised that mRNA uridylation may be a widespread gene regulatory mechanism with a potential role in oncogenesis. As the hypothesis was explored, Kim et al. reported that mRNA uridylation by ZCCHC6/11 globally regulates mRNA decay [8, 9], supporting this model and further highlighting the question of whether this mechanism plays a role in oncogenesis.

### ZCCHC6/11 are Expressed in Select Normal Tissues and Diverse Types of Cancer

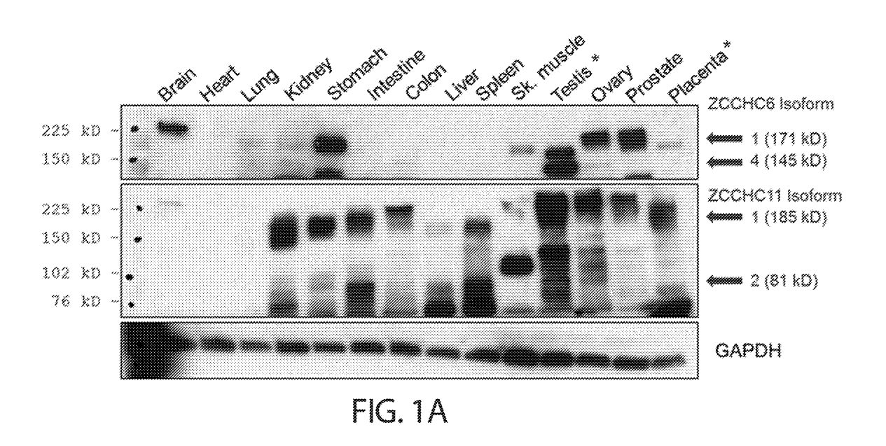

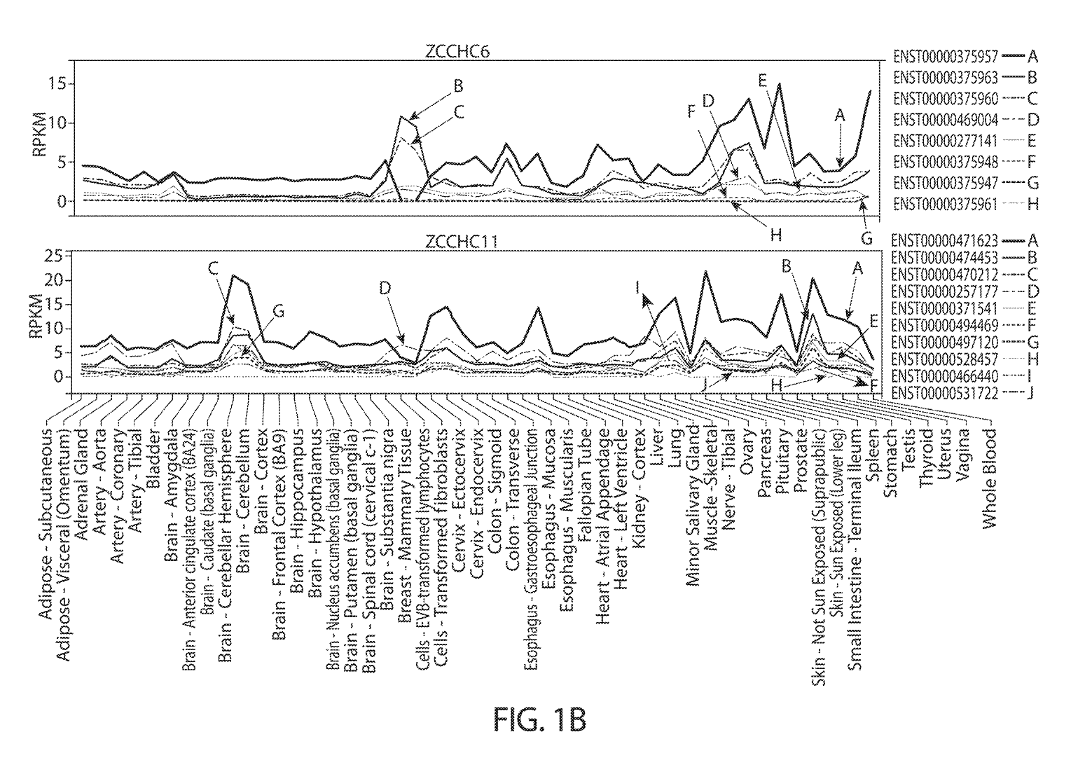

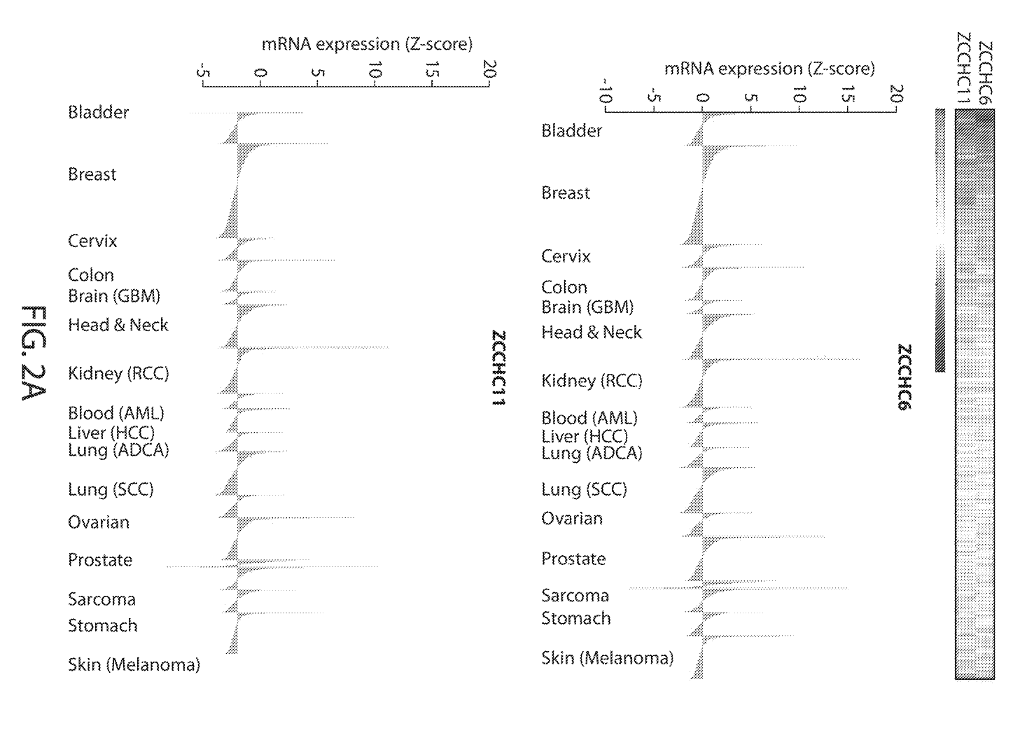

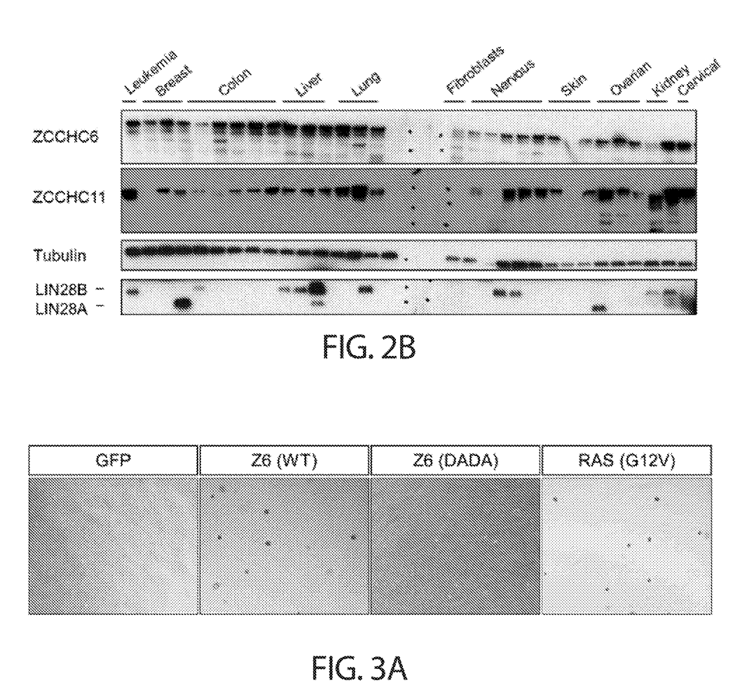

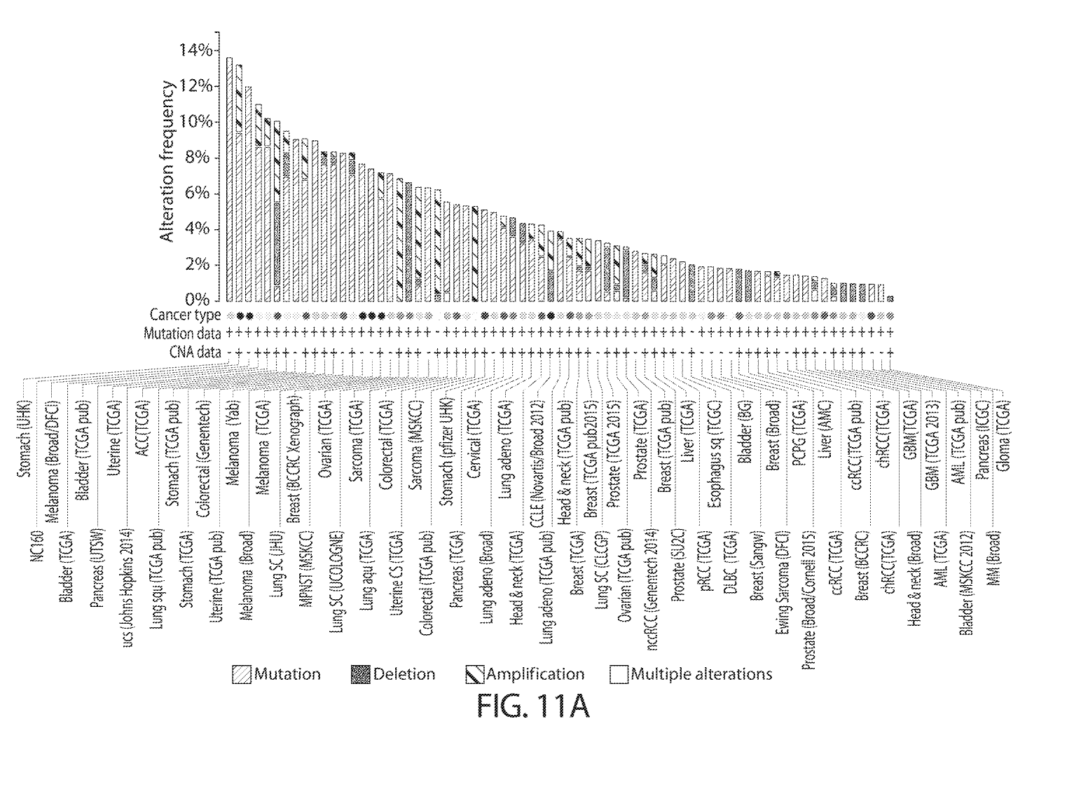

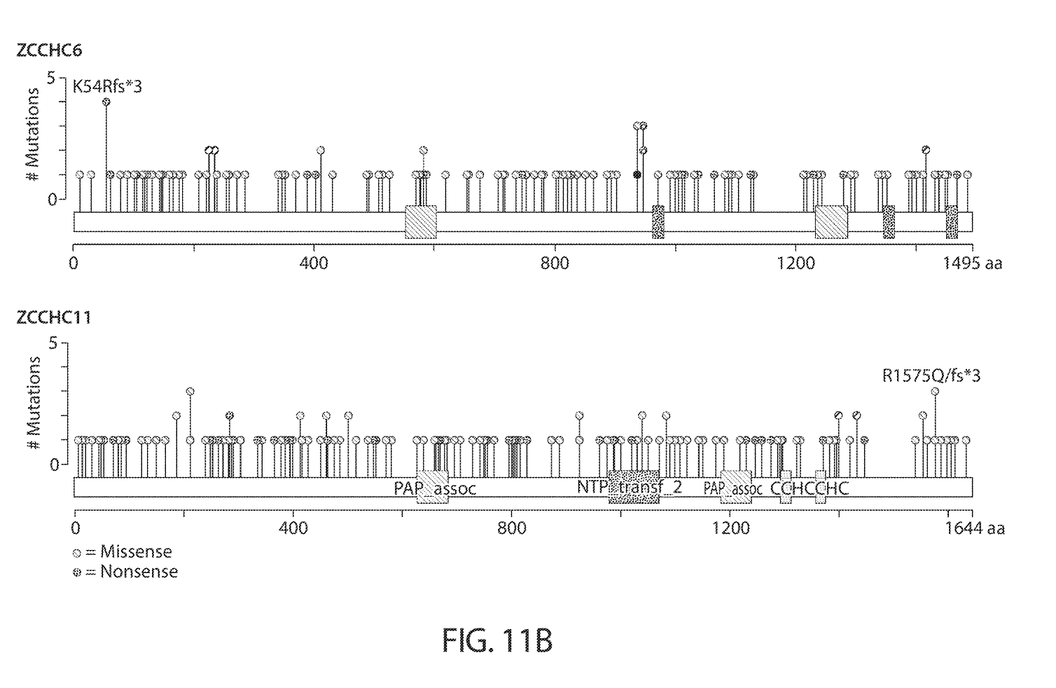

To explore the above question, the expression of ZCCHC6/11 was profiled across a range of normal and malignant contexts. First, a Western blot analysis was performed on a panel of 14 normal human tissues, which indicated that the TUTases—and particularly ZCCHC6—are expressed only in a subset of adult tissues (FIG. 1A). Publically available RNA-seq data revealed similar mRNA expression patterns (FIG. 1B). Of note, these TUTases have multiple predicted isoforms, only the largest of which (171 kD for ZCCHC6 and 185 kD for ZCCHC11) are known to be catalytically active and thus functional [6]. Hence, the present analysis was focused on these isoforms. Second, RNA-seq data was obtained from human patient samples comprising multiple distinct types of cancer, which revealed that about 30% of tumors across diverse cancers express high levels of ZCCHC6/11 (FIG. 2A). A panel of 30 cancer cell lines representing six different cancer types was assembled and analyzed via Western blot. Strikingly, the majority of cell lines expressed ZCCHC6 and/or ZCCHC11, with most exhibiting a much higher expression level relative to normal fibroblasts (FIG. 2B). Intersection of the normal-tissue and cancer expression data pointed to several tumor types that appear to express ZCCHC6/11 at a high level specifically in the cancer context (e.g. colon, lung, liver), indicating that these cancers may selectively upregulate the TUTases. Interestingly, a survey of available genomic data revealed low frequency of mutations, deletions, and amplifications associated with ZCCHC6/11, suggesting that transcriptional overexpression rather than genetic alterations affect TUTase expression and possibly function (FIGS. 11A-11B). Of note, ZCCHC6/11 expression did not correlate with LIN28A/B expression, pointing to LIN28-independent roles of the TUTases (FIGS. 1-2).

### TUTase Overexpression Promotes Oncogenic Transformation

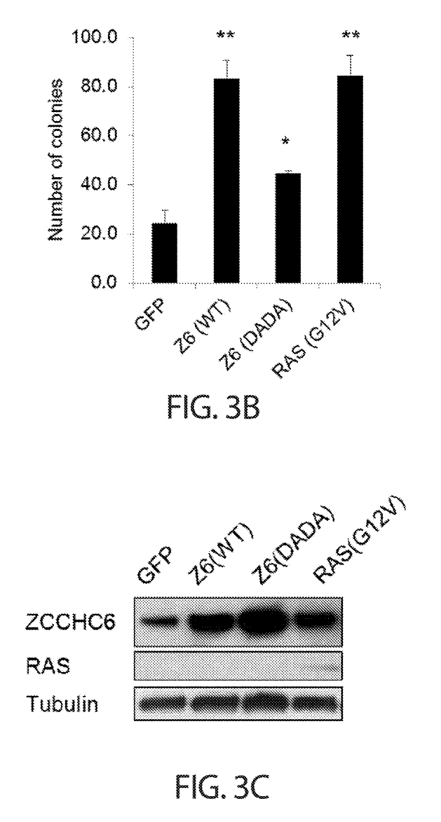

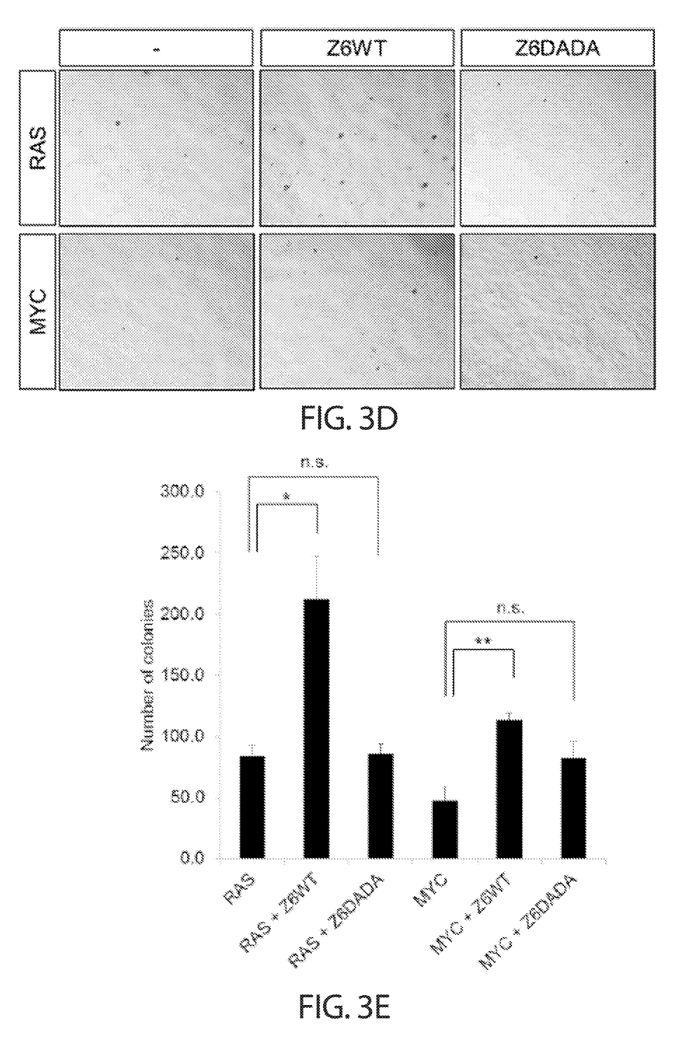

To determine if TUTase overexpression can promote oncogenic transformation, colony forming assays were performed in soft agar. Overexpression of wild-type ZCCHC6 led to an approximately four-fold increase in the number of colonies, which was comparable to the effect of mild expression of mutant RAS (G12V), suggesting that TUTase overexpression alone can be sufficient to induce oncogenic transformation (FIGS. 3A-3C). Importantly, mutating two critical aspartate residues to alanines (DADA), which renders ZCCHC6 catalytic-dead, largely abrogated its colony-forming ability, further indicating that its transforming function is mostly dependent on its enzymatic activity (FIGS. 3A-3C). Whether TUTase overexpression may enhance the transforming ability of classic oncogenes such as RAS and MYC was next examined. Wild-type or catalytic-null (DADA) ZCCHC6 with RAS (G12V) or MYC were co-overexpressed, which led to enhanced colony formation specifically in the case of the wild-type TUTase (FIGS. 3D-3E). Overall, these data suggest that TUTase overexpression can be sufficient to induce oncogenic transformation and may further cooperate with established oncogenes.

### TUTase Depletion Impairs Growth in Diverse Cancer Cell Types

Next, whether TUTase expression is required for the rapid growth of established cancer cells was determined. To this end, siRNA-based ZCCHC6/11 knockdowns were performed in a panel of 19 different cancer cell lines representing six distinct cancer types. Six of the cell lines—or about one-third—showed impaired growth upon TUTase depletion (FIG. 4A). They represented different types of cancer, which suggests that dependence on the TUTases is not associated with a defined class of cancer but rather a more universal feature of a subset of tumors across diverse cancer types (FIG. 4B). In addition, there was no correlation between TUTase dependence and LIN28A/B expression, indicating a novel, LIN28-independent role for ZCCHC6/11. To validate the knockdown data, a CRISPR/Cas9-based strategy was employed to genetically knock out ZCCHC6/11 in representative TUTase-dependent cell lines. As expected, the knockout resulted in impaired cell growth that mirrored the siRNA-based effects, confirming the validity of the observed phenotypes (FIGS. 10A-10B).

The impact of TUTase depletion in tumorigenic assays was then assessed. Using HCT116 colon cancer cells, which are dependent on ZCCHC6 but not ZCCHC11 (FIGS. 11A-11B), reduced focus formation was observed in adherent culture and colony-forming ability in soft agar upon ZCCHC6 knockout (FIGS. 4C-4D). These data suggest that TUTases support cancer cell growth under clonal density and anchorage-independent conditions, which serve as surrogates for tumorigenicity. To address tumorigenic properties in vivo, subcutaneous xenograft assays were performed in immunocompromised (Rag2−/−gc−/−) mice, which showed even greater degree of growth impairment in ZCCHC6 knockout vs. control tumors (FIGS. 4E-4G). Taken together, these results suggest that the TUTases support cancer cell growth and tumorigenicity in vitro and in vivo.

### TUTase Loss Sensitizes Cancer Cells to Disruption of RNA Metabolism

Given the observed phenotypes, whether there are conditions that specifically sensitize cancer cells to TUTase loss was examined. In particular, since TUTases have been associated with RNA turnover [9], it was surmised that they may play an especially important role in the context of perturbed RNA metabolism. To address this hypothesis, small molecule agents that are known to cause DNA/RNA damage and thus impair RNA quality (doxorubicin, cisplatin, etoposide), as well as ones that inhibit nucleotide synthesis and thereby limit the pool of available nucleotides in the cell (5-fluorouracil and hydroxyurea) [10] were selected, and wild-type and ZCCHC6 knockout HCT116 cells were treated with increasing concentrations of each agent (FIG. 5A). Of the tested drugs, only 5-fluorouracil (5-FU) yielded a differential dose-dependent response, having a reproducibly stronger effect on the knockout cells (FIG. 5B). Analogous analysis in a second cell line, H1299, led to similar results. These data indicate that TUTase loss sensitizes cancer cells to 5-FU but not DNA/RNA damage agents, suggesting that TUTase function may be particularly relevant to maintaining the flux of nucleotides during normal RNA turnover rather than clearance of damaged RNA molecules. Interestingly, hydroxyurea, which also impacts nucleotide metabolism, did not mirror the 5-FU results (FIG. 5B). 5-FU has been specifically shown to target the RNA exosome complex [11], which may explain its differential activity from hydroxyurea in the experiments. Overall, these results demonstrate that TUTase loss sensitizes cells to disruption of RNA metabolism, which highlights conditions of particular relevance to TUTase function and points to its underlying molecular mechanism of action.

TUTases Uridylate mRNAs and Enhance their Turnover in Cancer Cells

Next, the molecular basis of the observed phenotypes was elucidated. As TUTases have been shown to regulate both miRNAs and mRNAs [1, 3, 9, 12-14], whether the phenotypes are due to their miRNA-directed activity was first examined. Cell proliferation assays were performed using an isogenic pair of HCT116 cell lines that express wild-type DICER or are homozygous for a hypomorphic DICER allele (DICER^{Ex5}) and thus deficient in mature miRNAs [15]. Strikingly, the growth impairment upon ZCCHC6 knockdown was identical between the two cell lines, which suggests that ZCCHC6's effects are miRNA-independent and strongly implicates its mRNA-specific activity in the regulation of cell growth (FIGS. 11A-11B).

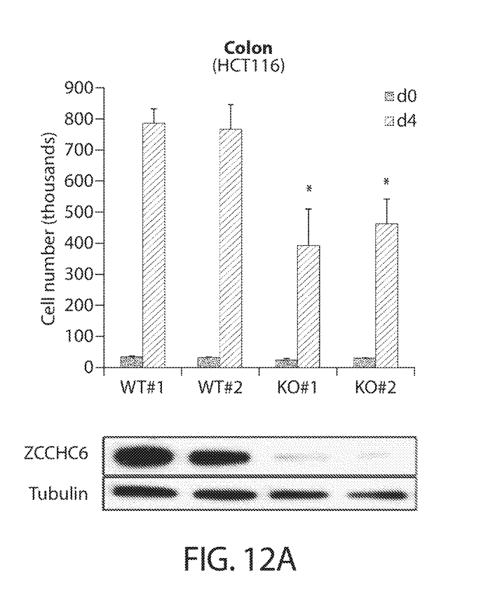

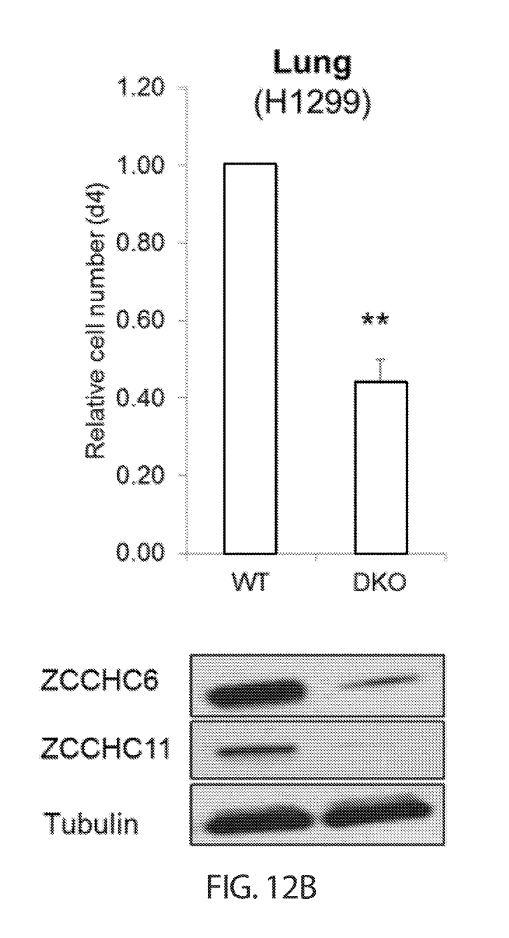

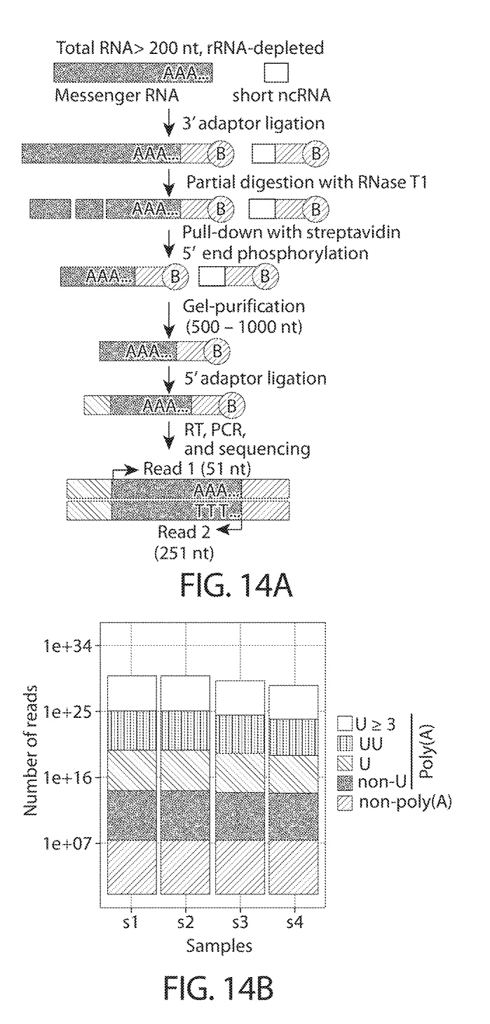

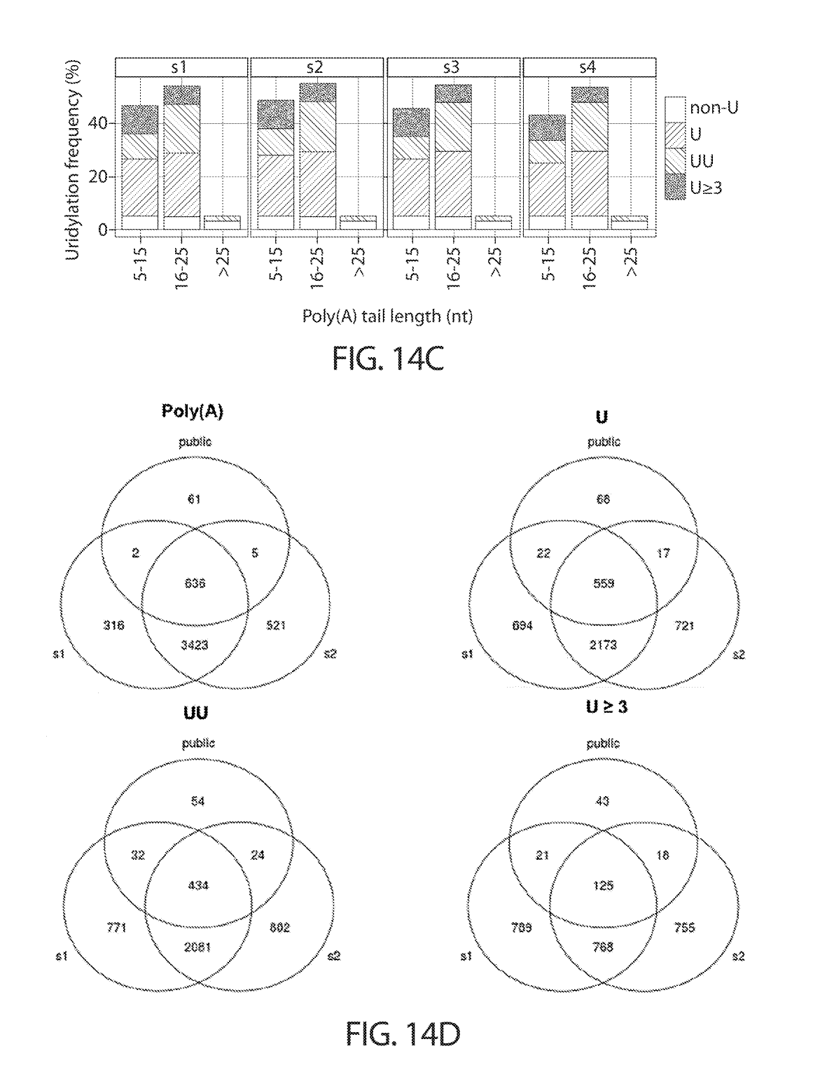

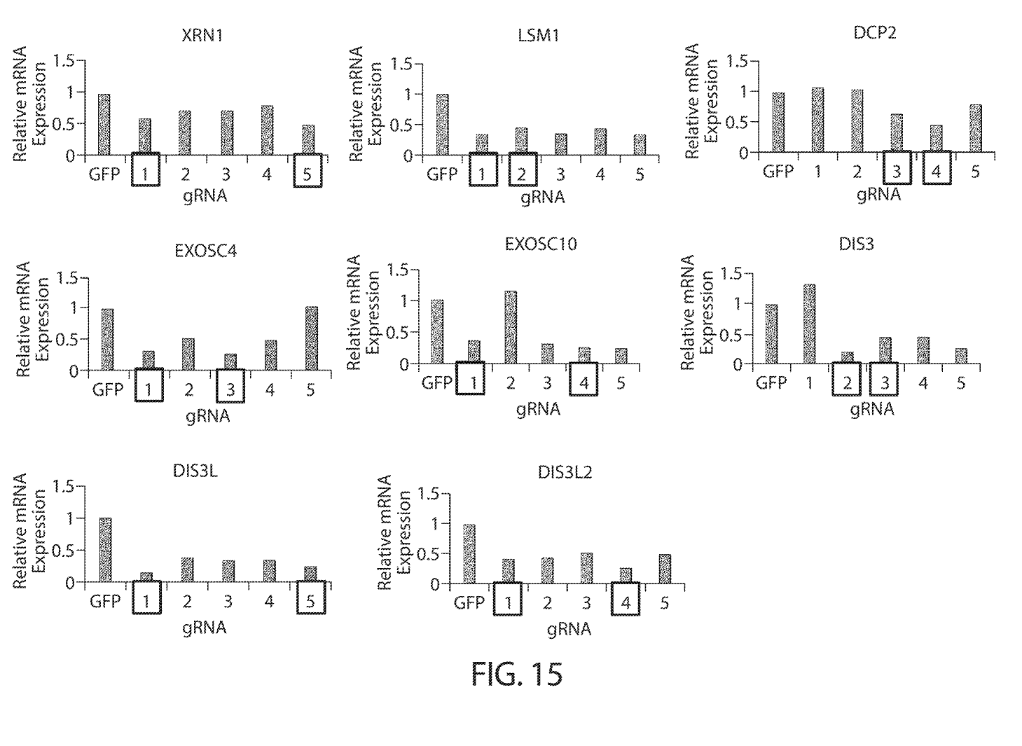

To assess mRNA-based TUTase activity, the recently developed TAIL-seq method for genome-wide investigation of the 3′ mRNA terminome was used (FIG. 14A) [8]. After optimizing most of the protocol steps, pilot TAIL-seq analyses in HeLa cells were performed using four different iterations of the optimized protocol. HeLa cells were used for these pilot runs since the public TAIL-seq data were obtained from this cell type [8], thus allowing a direct assessment of how the adapted protocol performs in comparison to the published one. Overall, the data closely resembled the published analyses to date [8, 9], validating the TAIL-seq protocol in the present experiment (FIGS. 12B-12D).

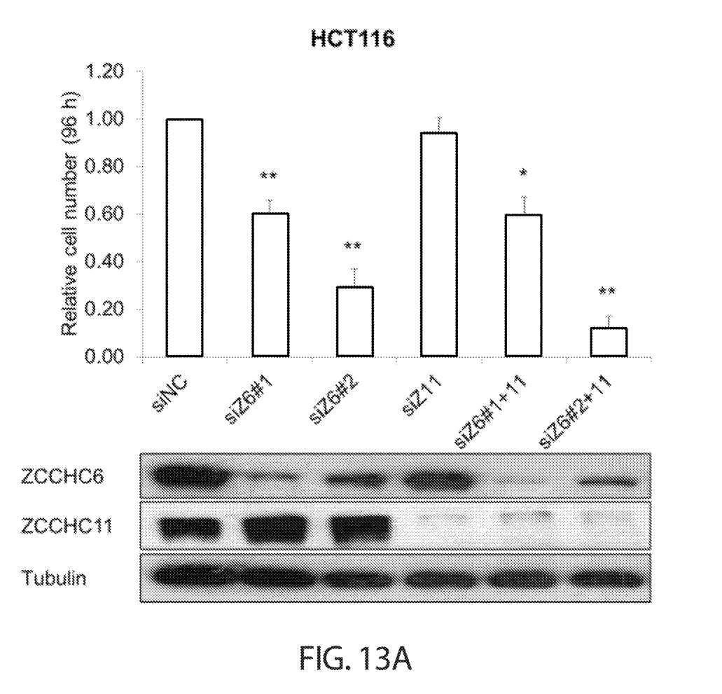

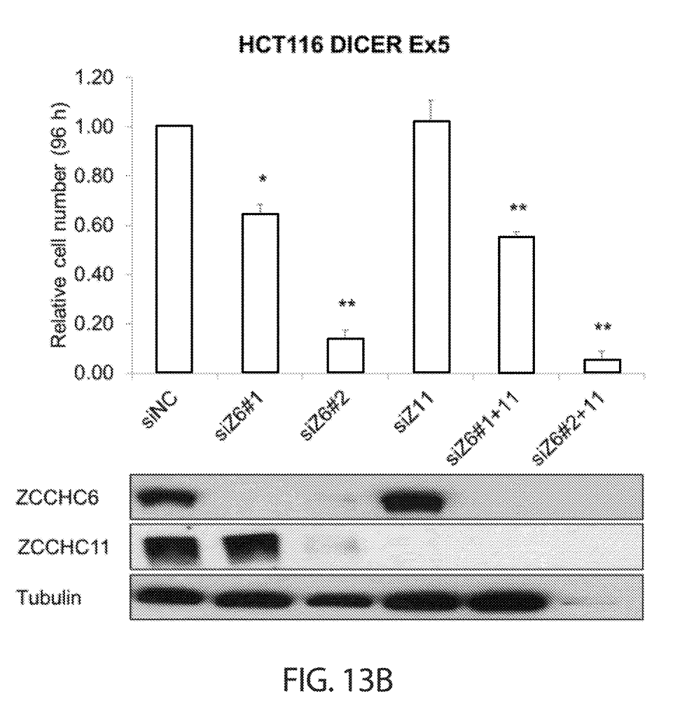

TAIL-seq was then applied to HCT116 cells after ZCCHC6 depletion. Consistent with ZCCHC6's molecular function, mRNA uridylation was globally reduced in the knockdown versus the control condition, indicating that ZCCHC6 uridylates mRNAs in these cells (FIG. 6A). To examine if the decreased uridylation impacts mRNA levels, analogous knockdown experiments were performed, followed by RNA-seq on large RNA (>200 nt) fractions. Interestingly, mRNA levels were largely unchanged, suggesting that TUTase depletion has a limited effect on steady-state mRNA levels (FIG. 6B). Of note, two alternative library preparation strategies, polyA-selection and rRNA depletion (RiboZero), were used to avoid potential bias against the preferentially uridylated short polyA tails in the more commonly used polyA-selection protocol. Both strategies yielded essentially the same results, ruling out transcript bias as the reason for lack of considerable changes in mRNA levels (FIG. 6B). Since steady-state mRNA measurements cannot readily uncover effects on mRNA turnover and uridylation negatively correlates with mRNA half-life (FIG. 6C), mRNA half-life measurements were performed after ZCCHC6 depletion using actinomycin D chase coupled with RNA-seq (FIG. 6D). This analysis revealed a genome-wide increase of mRNA half-life in ZCCHC6 knockdown vs. control cells, indicating that ZCCHC6 is required for global maintenance of rapid mRNA decay and thereby turnover (FIG. 6D). Half-life measurements of representative transcripts were further validated using qRT-PCR analysis (FIG. 6E). Lastly, half-life measurements of selected short non-coding RNAs (sncRNAs) were performed under the same experimental conditions, which did not show a difference between ZCCHC6 knockdown and control cells (FIG. 6E). Together with the DICER^{Ex5} data (FIGS. 13A-13B), these results further point to mRNAs (or other polyA RNAs) rather than sncRNAs as functionally relevant TUTase targets, at least in this context. Overall, the findings demonstrate that TUTases uridylate mRNAs and enhance their turnover in cancer cells.

### In Vivo Assessment of the Role of TUTases in Tumorigenesis

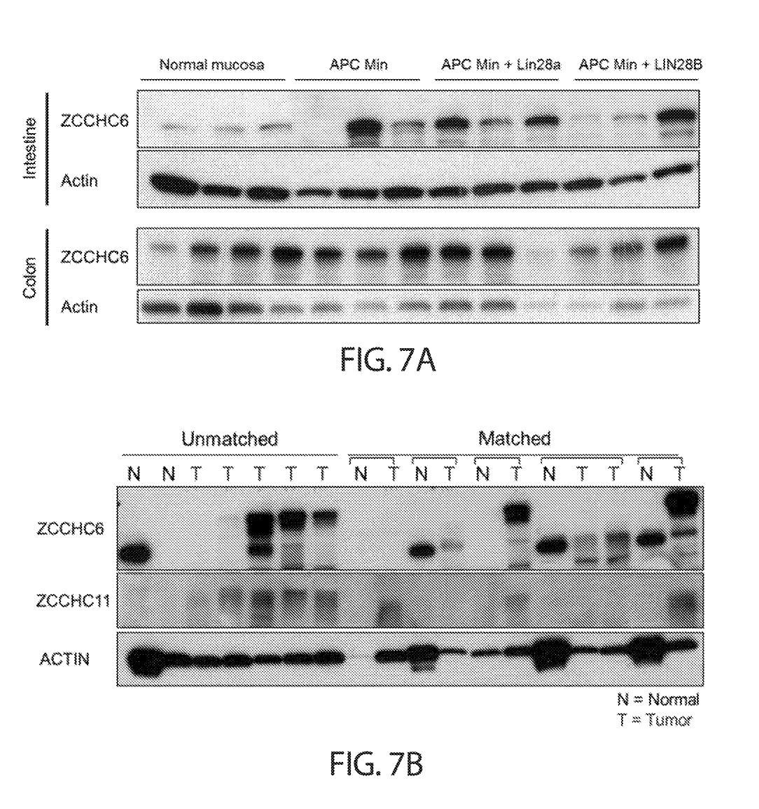

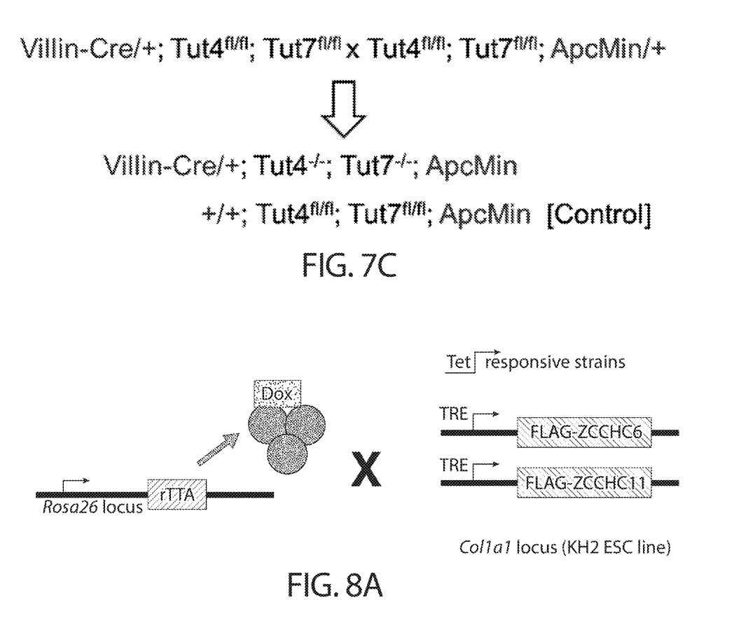

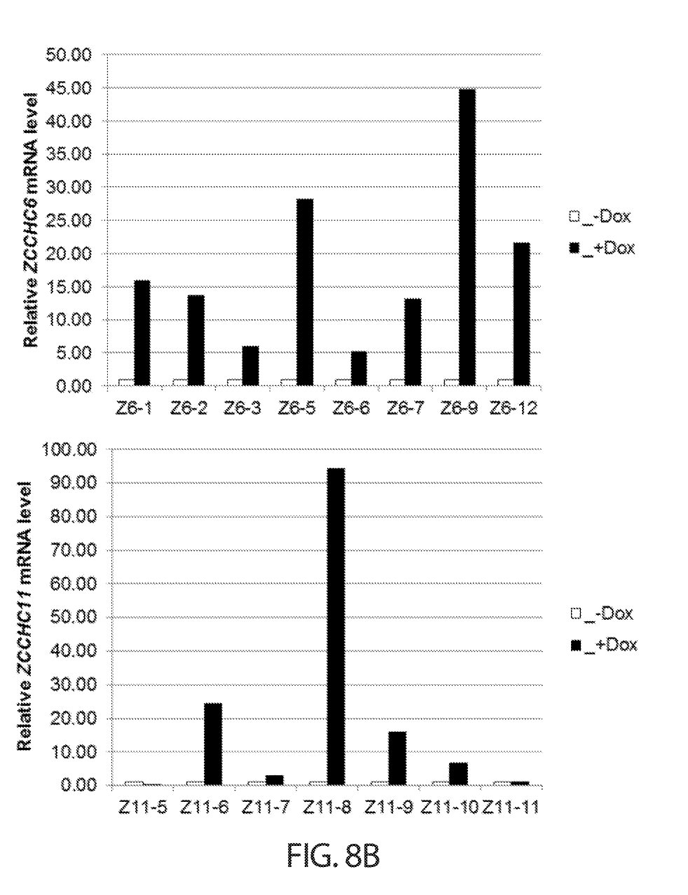

To explore the role of TUTases in oncogenesis in vivo, Zcchc6/Tut7 and Zcchc11/Tut4 conditional knockout mice were obtained and bred into established mouse models of cancer (colon and Wilms). Colon cancer was first examined, since a Western blot analysis of tissue samples from the ApcMin, ApcMin+Lin28a, and ApcMin+LIN28B mouse models of colon cancer revealed higher Zcchc6 levels in the tumors relative to normal mucosa (FIG. 7A). In addition, analysis of tissue lysates from human colorectal tumors showed similar reactivation of ZCCHC6/11 in a considerable number of cases (5/11 samples analyzed) (FIG. 7B). Hence, a genetic strategy to assess the impact of TUTase loss on ApcMin-driven tumorigenesis by employing intestine-specific VillinCre was designed to yield tissue-restricted Zcchc6/11 double knockout (FIG. 7C). Lastly, to examine if TUTase overexpression may promote tumorigenesis in vivo, doxycycline-inducible ZCCHC6 (iZ6) and ZCCHC11 (iZ11) mESCs were generated, and successful ZCCHC6/11 overexpression was validated via qRT-PCR in individual clones, which can be used for mouse generation after additional validation (FIGS. 8A-8C).

### TUTase Loss has a Downstream Impact on Protein Turnover

Given that TUTases affect mRNA turnover, it was also surmised that they may have a downstream effect on protein turnover. To address this hypothesis, the global rate of translation in TUTase knockdown versus control HCT116 cells was measured using the OP-Puromycin method [23]. TUTase depletion led to an approximate 30% reduction in translation rate, suggesting that TUTase-mediated regulation of mRNA turnover is coupled with protein synthesis (FIG. 16). In addition, we treated wild-type and TUTase knockout HCT116 cells with increasing concentrations of bortezomib, an inhibitor of the proteasome and thus protein decay (FIG. 16). The effect on cell viability was stronger in the TUTase knockout cells, indicating that TUTase loss sensitizes cancer cells to disruption of protein turnover. Taken together, these data suggest that TUTases' effects on mRNA turnover have a downstream impact on protein turnover.

### REFERENCES

1. Heo, I., et al., Mono-uridylation of pre-microRNA as a key step in the biogenesis of group II let-7 microRNAs. Cell, 2012. 151(3): p. 521-32.
2. Piskounova, E., et al., Lin28A and Lin28B inhibit let-7 microRNA biogenesis by distinct mechanisms. Cell, 2011. 147(5): p. 1066-79.
3. Thornton, J. E., et al., Lin28-mediated control of let-7 microRNA expression by alternative TUTases Zcchc11 (TUT4) and Zcchc6 (TUT7). RNA, 2012. 18(10): p. 1875-85.
4. Suzuki, H. I., A. Katsura, and K. Miyazono, A role of uridylation pathway for blockade of let-7 microRNA biogenesis by Lin28B. Cancer Science, 2015. 106(9): p. 1174-81.

5. Mullen, T. E. and W. F. Marzluff, Degradation of histone mRNA requires oligouridylation followed by decapping and simultaneous degradation of the mRNA both 5′ to 3′ and 3′ to 5′. Genes & Development, 2008. 22(1): p. 50-65.
6. Rissland, O. S., A. Mikulasova, and C. J. Norbury, Efficient RNA polyuridylation by noncanonical poly(A) polymerases. Molecular and Cellular Biology, 2007. 27(10): p. 3612-24.
7. Rissland, O. S. and C. J. Norbury, Decapping is preceded by 3′ uridylation in a novel pathway of bulk mRNA turnover. Nature Structural & Molecular Biology, 2009. 16(6): p. 616-23.
8. Chang, H., et al., TAIL-seq: genome-wide determination of poly(A) tail length and 3′ end modifications. Molecular Cell, 2014. 53(6): p. 1044-52.
9. Lim, J., et al., Uridylation by TUT4 and TUT7 Marks mRNA for Degradation. Cell, 2014. 159(6): p. 1365-76.
10. Wurtmann, E. J. and S. L. Wolin, RNA under attack: cellular handling of RNA damage. Critical Reviews in Biochemistry and Molecular Biology, 2009. 44(1): p. 34-49.
11. Kammler, S., S. Lykke-Andersen, and T. H. Jensen, The RNA exosome component hRrp6 is a target for 5-fluorouracil in human cells. Molecular Cancer Research: MCR, 2008. 6(6): p. 990-5.
12. Thornton, J. E., et al., Selective microRNA uridylation by Zcchc6 (TUT7) and Zcchc11 (TUT4). Nucleic Acids Research, 2015. 42(18): p. 11777-91.
13. Wyman, S. K., et al., Post-transcriptional generation of miRNA variants by multiple nucleotidyl transferases contributes to miRNA transcriptome complexity. Genome Research, 2011. 21(9): p. 1450-61.
14. Liu, X., et al., A MicroRNA precursor surveillance system in quality control of MicroRNA synthesis. Molecular Cell, 2014. 55(6): p. 868-79.
15. Cummins, J. M., et al., The colorectal microRNAome. Proceedings of the National Academy of Sciences of the United States of America, 2006. 103(10): p. 3687-92.
16. Lubas, M., et al., Exonuclease hDIS3L2 specifies an exosome-independent 3′-5′ degradation pathway of human cytoplasmic mRNA. The EMBO Journal, 2013. 32(13): p. 1855-68.
17. Malecki, M., et al., The exoribonuclease Dis3L2 defines a novel eukaryotic RNA degradation pathway. The EMBO Journal, 2013. 32(13): p. 1842-54.
18. Thomas, M. P., et al., Apoptosis Triggers Specific, Rapid, and Global mRNA Decay with 3′ Uridylated Intermediates Degraded by DIS3L2. Cell Reports, 2015. 11(7): p. 1079-89.
19. Schoenberg, D. R. and L. E. Maquat, Regulation of cytoplasmic mRNA decay. Nature Reviews Genetics, 2012. 13(4): p. 246-59.
20. Wright, J. E. and R. Ciosk, RNA-based regulation of pluripotency. Trends in Genetics: TIG, 2013. 29(2): p. 99-107.
21. Ye, J. and R. Blelloch, Regulation of pluripotency by RNA binding proteins. Cell Stem Cell, 2014. 15(3): p. 271-80.
22. Audic, Y. and R. S. Hartley, Post-transcriptional regulation in cancer. Biology of the Cell/under the auspices of the European Cell Biology Organization, 2004. 96(7): p. 479-98.
23. Liu, J. et al., Imaging protein synthesis in cells and tissues with an alkyne analog of puromycin. Proc Natl Acad Sci USA. 2012 Jan. 10; 109(2):413-8.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

### EQUIVALENTS AND SCOPE

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A method of treating cancer, the method comprising administering to a subject in need thereof a composition comprising a therapeutically effective amount of a terminal uridylyl transferase (TUTase) inhibitor to treat the cancer, wherein the cancer does not express LIN28A/B.

2. The method of 1, wherein the TUTase is ZCCHC11 or ZCCHC6.

3. The method of 1, wherein the TUTase inhibitor is a small molecule, an oligonucleotide, an antibody, or an antibody fragment.

4. The method of 3, wherein the TUTase inhibitor inhibits the TUTase's enzymatic activity.

5. The method of claim 4, wherein the TUTase inhibitor is selected from the group consisting of: SCH 202676 hydrobromide, Tryphostin 47, FPA 124, Ebselen, Aurothioglucose hydrate, and IPA-3.

6. The method of claim 3, wherein the TUTase inhibitor reduces TUTase expression.

7. The method of claim 6, wherein the TUTase inhibitor is RNAi targeting ZCCHC11 or ZCCHC6, or a vector that co-expresses a Cas9 nuclease, and a guide RNA that targets the Cas9 to the ZCCHC11 or ZCCHC6 gene, whereby the ZCCHC6 or ZCCHC11 gene is cleaved by the Cas9.

8. (canceled)

9. The method of claim 3, wherein the antibody or the antibody fragment is specific to ZCCHC11 or ZCCHC6.

10. The method of claim 1, wherein the composition further comprises a therapeutically effective amount of an agent that disrupts RNA metabolism.

11. The method of claim 10, wherein the agent that disrupts RNA metabolism inhibits nucleotide synthesis or metabolism.

12.-18. (canceled)

19. The method of claim 1, wherein the composition further comprises an agent that disrupts protein metabolism.

20. The method of claim 19, wherein the agent that disrupts protein metabolism inhibits protein turnover.

21. The method of claim 20, wherein the agent that disrupts protein metabolism is a proteasome inhibitor.

22.-28. (canceled)

29. The method of claim 1, wherein the composition inhibits cancer cell growth, reduces tumor size, and/or prevents metastasis.

30. (canceled)

31. The method of claim 1, wherein the composition is administered via injection to the cancer or is administered systemically.

32. (canceled)

33. The method of claim 1, wherein the subject is a mammal.

34.-38. (canceled)

39. A method of treating cancer, the method comprising administering to a subject in need thereof a composition comprising a therapeutically effective amount of a terminal uridylyl transferase (TUTase) inhibitor and an agent that disrupts RNA metabolism, to treat the cancer.

40.-65. (canceled)

66. The method of claim 39, wherein the cancer does not express LIN28A/B.

67.-76. (canceled)

77. A pharmaceutical composition comprising a terminal uridylyl transferase (TUTase) inhibitor for treating cancer, wherein the cancer does not express LIN28A/B.

78. A pharmaceutical composition comprising a terminal uridylyl transferase (TUTase) inhibitor and an agent that disrupts RNA metabolism, for treating cancer.

79.-84. (canceled)


SEQUENCE LISTING (1 sequence)

SEQ ID NO: 1 - protein, 4 aa, Artificial Sequence
  Synthetic Polypeptide
DADA